(19) United States
(12) Patent Application Publication
(10) Pub. No.: US 2007/0196834 A1
(43) Pub. Date: Aug. 23, 2007

(54) Method and system for the generation of large double stranded DNA fragments

(76) Inventors: Francesco Cerrina, Madison, WI (US); James H. Kaysen, Madison, WI (US); Mo-Huang Li, Singapore (SG); Larry Li-Yang Chu, Stafford, TX (US); Peter J. Belshaw, Madison, WI (US); Michael R. Sussman, Madison, WI (US); Kathryn Richmond, Madison, WI (US)

Correspondence Address: FOLEY & LARDNER LLP, 150 EAST GILMAN STREET, P.O. BOX 1497, MADISON, WI, 53701-1497, US

(21) Appl. No.: 11/506,150

(22) Filed: Aug. 17, 2006

Related U.S. Application Data
(60) Provisional application No. 60/715,623, filed on Sep. 9, 2005.

(51) Int. Cl. C12Q 1/68; C12P 19/34; C07H 21/04

(52) U.S. Cl. 435/6; 435/91.2; 536/25.3

(57) ABSTRACT

Synthesis of long chain molecules such as DNA is carried out rapidly and efficiently to produce relatively large quantities of the desired product. The synthesis of an entire gene or multiple genes formed of many hundreds or thousands of base pairs can be accomplished rapidly and, if desired, in a fully automated process requiring minimal operator intervention, and in a matter of hours, a day or a few days rather than many days or weeks. Production of a desired gene or set of genes having a specified base pair sequence is initiated by analyzing the specified target sequence and determining an optimal set of subsequences of base pairs that can be assembled to form the desired final target sequence. The set of oligonucleotides are then synthesized utilizing automated oligonucleotide synthesis techniques. The synthesized oligonucleotides are subsequently selectively released from the substrate and used in a sequential assembly process.

### CROSS-REFERENCE TO RELATED APPLICATION

This application claims the benefit of provisional patent application No. 60/715,623, filed Sep. 9, 2005, the disclosure of which is incorporated herein by reference.

### STATEMENT OF GOVERNMENT RIGHTS

This invention was made with United States government support awarded by the following agency: DOD ARPA DAAD 19-02-2-0026. The United States government has certain rights in this invention.

### FIELD OF THE INVENTION

The present invention relates generally to the field of molecular biology and particularly to the artificial synthesis of long DNA fragments including fragments encompassing a gene or multiple genes.

### BACKGROUND OF THE INVENTION

Significant efforts have been made to synthesize genes from oligonucleotides, with the assembly of viral and bacteriophage genomes being reported. See, e.g., J. Cello, et al., Science, 297, 2002, pp. 1016-1018; H. O. Smith, et al., Proc. Natl. Acad. Sci. USA, 100, 2003, pp. 15440-15445. Assembly of these long sequences required the use of hundreds of commercially synthesized and gel-purified olignucleotides. Thus, such approaches are not economically feasible for the routine synthesis of genes for research and clinical purposes.

Over the last decade, techniques have been developed for the synthesis of DNA (deoxyribonucleic acid) on solid substrates for use in genetics studies, particularly for hybridization experiments with microarrays. These developments have included systems to carry out precision patterning and fluorescence analysis. See, e.g., P. B. Garland, et al., Nucleic Acids Res., 30, 2002, pp. e99, et seq: A. Relogio, et al., Nucleic Acids Res., 30, 2002, pp. e51 et seq. DNA “chips” formed in this manner offer the potential for acquiring a large number of user-defined DNA oligonucleotide sequences for subsequent use in biological applications. Although oligonucleotides grown on slide surfaces have been extensively employed in this manner, there remains some uncertainty concerning the amount and relative proportion of failure sequences on the chip surface. Previous studies have estimated that a total of about 10 to 30 pmol/cm² of oligonucleotides are synthesized on the chip surface. G. McGall, et al., J. Am. Chem. Soc., 119, 1997, pp. 5081-5090; E. LeProust, et al., Nucleic Acids Res., 29, 2001, pp. 2171-2180. However, it is not clear whether this estimate represents the population of full-length product or a mixture of full-length and truncated or mutated sequences. In studies using photogenerated acids during DNA synthesis, it has been postulated that proximity to the synthesis surface led to lower fidelity, and that this decrease is due to inefficient reactions of various reagents. It is unclear, however, whether such surface effects occur in photolithographic procedures using photolabile 2-nitrophenyl propoxycarbonyl (NPPOC) photodeprotection-based DNA synthesis.

Historically, scientists have made use of gene synthesis to produce those genes recalcitrant to cloning due to high organismal A-T or G-C content or to modify genes for optimal protein expression and heterologous hosts. Such expression targets are generally less than three thousand bp (base pairs) in length. Gene synthesis has also been utilized to create larger assemblages (e.g., 7-8 kb) but the conventional techniques used have often required very long lengths of time (e.g., months) to obtain the final product. J. Cello, supra.

New techniques have been developed for the assembly of genes, including ligase-chain reaction (LCR) and suites of polymerase chain reaction (PCR) strategies. While most gene assembly protocols start with pools of overlapping synthesized oligonucleotides, and end with PCR amplification of the assembled gene, the pathway between those two points can be quite different. In the case of LCR, the initial oligonucleotide population is required to have phosphorylated 5 ends that allow Pfu DNA ligase to covalently connect these building blocks together to form the initial template. Single stranded (ss) PCR assembly, however, makes use of unphosphorylated oligonucleotides, which undergo repetitive PCR cycling to extend and create a fill length template. A variant of this method, termed double stranded (ds) PCR involves combining all single stranded PCR oligonucleotides and their reverse complement oligonucleotides for assembly. Additionally, the LCR process requires oligonucleotide concentrations in the μM(10⁻⁶) range, whereas both ss and ds PCR options have concentration requirements that are much lower (nM, 10⁻⁹ range). The relative efficiencies and mutation rates inherent in these different strategies are not necessarily well understood. In addition to the manner used to assemble genes, the size of the initial oligonucleotides utilized may also have significant impact upon the final product and the efficiency of the process. Prior synthesis attempts have generally used oligonucleotides ranging in size from 20 to 70 bp, assembled through hybridization of overlaps in the range of 6-40 bp. Since many factors in the process are determined by the length and composition of the oligonucleotides (Tₘ, secondary structure, etc.), the size and heterogeneity of the initial oligonucleotide population can have a significant effect on the efficiency of the assembly and the quality of the final assembled genes.

### SUMMARY OF THE INVENTION

In accordance with the present invention, synthesis of long chain molecules such as DNA is carried out rapidly and efficiently to produce relatively large quantities of the desired product. The synthesis of an entire gene or multiple genes formed of many hundreds or thousands of base pairs can be accomplished rapidly and, if desired, in a fully automated process requiring minimal operator intervention, and in a matter of a day or a few days rather than many days or weeks.

In the present invention, production of a desired gene or set of genes having a specified base pair sequence is initiated by analyzing the specified target sequence and determining a set of subsequences of base pairs that can be assembled to form the desired final target sequence. For example, a target sequence having several hundreds or thousands of base pairs may be divided up into a set of subsequences each having a much smaller number of base pairs, e.g., 400 to 600 bp, which are then further divided into oligonucleotide sequences, e.g., in the range of 20 to 100 bp, which may be conveniently synthesized utilizing automated oligonucleotide synthesis techniques. An exemplary oligonucleotide synthesis technique utilizes a maskless array synthesizer (MAS) by which large numbers of different oligonucleotide sequences (e.g., 50 to 100 bases in length) are generated in a array on a support in a few hours under computer control utilizing phosphoramidite chemistry without moving parts or operator intervention, although other synthesis materials and techniques may also be utilized. The synthesized oligonucleotides are subsequently selectively released from the support to be used in a sequential assembly process. The oligonucleotides may be released utilizing, for example, base labile linkers or photo-cleavable linkers. In a preferred process, the oligonucleotide sequences include not only the desired subsequences for the final product but also end sequences that may be utilized as primers in the polymerase chain reaction (PCR), allowing the initial set of oligonucleotides to be greatly amplified in volume using PCR techniques. After the oligonucleotides have been amplified by PCR, the primer sequences are then removed, leaving only the desired oligonucleotides.

DNA error filtering is preferably carried out on short double-stranded oligonucleotides and longer DNA fragments before and during the assembly process. An exemplary error filtering technique is DNA coincidence filtering, which utilizes the bacterial MutS protein to bind DNA duplexes containing mismatched bases while allowing error free duplexes to pass through. Assembly chambers are utilized for mixing and thermal cycling during the DNA fragment assembly. Oligonucleotides or intermediate sized DNA fragments flow into the chambers along with PCR buffer, deoxynucleotide triphosphates, and thermostable DNA polymerase. These reagents are then mixed, e.g. by ultrasonic mixing, and then thermal cycled for assembly and amplification reactions. An integrated fluidic system collects the released oligonucleotides from the synthesis chamber and routes them through the error filters to and from the assembly chambers. The system also delivers reagents needed for fragment assembly and error filtering. The fluidic system is preferably constructed of microfluidic channels and includes integrated micro-valves, flow sensors, heaters, ultrasonic mixers, and appropriate connections to external reagents, pumps and waste containers.

Further objects, features and advantages of the invention will be apparent from the following detailed description when taken in conjunction with the accompanying drawings.

### BRIEF DESCRIPTION OF THE DRAWINGS

In the drawings:

FIG. 1 is a simplified summary diagram of the gene assembly process of the invention.

FIG. 2 is a simplified diagram illustrating the gene fabrication process sequence in accordance with the invention.

FIG. 3 is a schematic illustration of the safety catch photoliable linker process that may be utilized in the invention.

FIG. 4 are chemical diagrams illustrating phosphoramidites which may be used for base labile linker chemistry.

FIG. 5 are chemical diagrams illustrating the synthesis of acid-activated safety catch photolabile linker.

FIG. 6 are chemical diagrams of photolabile protecting groups NPPOC (1.0), (8NNa) MOC (1.5), and 5 (2Na) NPPOC (3.0) (relative deprotection rates shown in parenthesis) for use in DNA synthesis.

FIG. 7 is a graph illustrating the performance of various sensitizer molecules in deprotecting NPPOCT at wavelengths longer than 400 nm.

FIG. 8 are chemical diagrams illustrating a synthesis of base-activated SCPL-linker.

FIG. 9 is a schematic diagram illustrating the consensus filtering process.

FIG. 10 is a diagrammatic representation of an illumination and optical system of a maskless array synthesizer that may be utilized in the invention.

FIG. 11 is a schematic diagram of a image locking system in the maskless array synthesizer of FIG. 10.

FIG. 12 is a diagrammatic representation of a reference mark on a reaction cell.

FIG. 13 is a diagrammatic representation of a projected alignment pattern on a glass slide.

FIG. 14 is a diagrammatic representation of locations of alignment marks.

FIG. 15 is a simplified cross-sectional view of a reaction cell with image locking.

FIG. 16 is a diagrammatic representation of a captured image to be processed in the maskless array synthesizer.

FIG. 17-19 are examples of captured images to be processed.

FIG. 20 is a diagrammatic representation of a image projected on a substrate wherein the image includes several micromirrors.

FIG. 21 is a schematic diagram of the manner of appearance of the micromirrors in the field of a microscope with respect to the maskless array synthesizer.

FIG. 22 is a simplified cross-sectional view of a synthesis cell incorporating microspheres in the reaction chamber.

FIG. 23 has a partially schematic view of a capillary tube apparatus for use in synthesis of chain molecules.

FIG. 24 is a simplified diagram illustrating the steps in the process of the assembly of genes including the post-synthesis fluid handling steps performed in a repetitive manner.

FIG. 25 is a illustrative diagram of a post-processing system using robotics and micropipettes.

FIG. 26 is a simplified cross-sectional view of a modified pipette tip with integrated MutS filtering element for parallel error-filtering.

FIG. 27 is a diagrammatic view illustrating steps in the basic process of forming a microfluidic handling system.

FIG. 28 is a schematic view of an integrated post-synthesis processing system.

FIG. 29 is a flow diagram illustrating the control steps carried out in process monitoring.

FIG. 30 is a schematic diagram illustrating light directed combinatorial synthesis, in which a substrate is coated with a scaffold molecule protected with a photolabile protecting group (PL) and additional latent photocleavable protecting groups (PGx).

FIG. 31 are chemical diagrams illustrating the activation of safety catch and photo cleavage of long wavelength trimethoxyphenacyl protecting groups.

FIG. 32 are chemical diagrams illustrating a synthesis route for safety-catch photo cleavable protecting groups.

FIG. 33 are chemical diagrams illustrating the synthesis of test compounds.

FIG. 34 are chemical diagrams illustrating the synthesis of a SCPL-protected Lys-Ser scaffold.

### DETAILED DESCRIPTION OF THE INVENTION

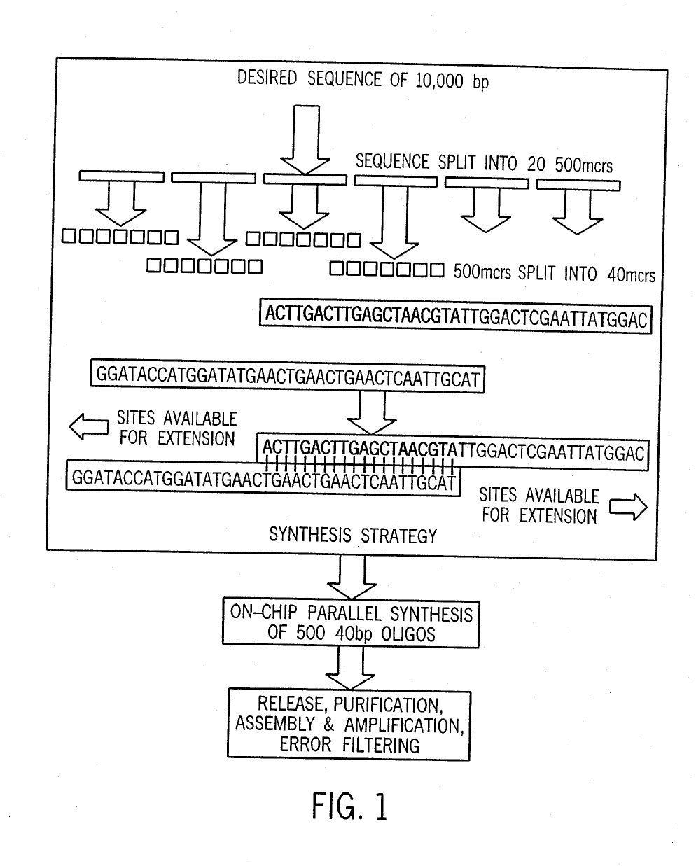

For purposes of exemplifying the invention, FIG. 1 illustrates in summary form a process by which a desired target sequence of, e.g., ten thousand base pairs (bp) forming a desired set of genes can be synthesized. It is understood that this example is provided as a representative case, and that the invention is not limited to such examples. To develop the synthesis strategy (using bioinformatics computer software algorithms as discussed further below), the desired target sequence is analyzed and split (for the 10,000 bp example) into 20 intermediate sequences of 500 bp each, and the 500 bp intermediate sequences are then split into a total of 500 subsequences of 40 bp (25 subsequences for each intermediate sequence), which are lengths that can be conveniently synthesized using automated oligonucleotide synthesis techniques. After the synthesis strategy has been developed, parallel synthesis of the 500 specified 40 bp oligonucleotides is carried out, followed by selectively sequential release of the oligonucleotides, purification, assembly and amplification, and error filtering. It should be understood that the length of the assembly blocks can be selected as desired and the lengths of the blocks can be individually varied to optimize the process.

An exemplary oligonucleotide synthesis system in accordance with the invention uses the intrinsic parallelism of optical imaging that allows very high densities (>300,000 cm⁻²) of oligonucleotide sequences to be synthesized on a support such as a glass surface. By releasing selected oligonucleotides from the support in an effective and controllable way, long dsDNA can be created by assembling the short oligonucleotide pieces. Thus, after release and step-wise assembly, the desired dsDNA sequence is formed. The gene assembly system is thus based on four capabilities: (1) the ability to synthesize arbitrary sequences of short oligomers in a massively parallel way, in situ, starting from monomers; (2) the ability to selectively release from the synthesis support whichever oligomer sequences are desired in order to perform a partial assembly; (3) the ability to assemble these intermediate length oligomers into a full length final product; and (4) the ability to filter and eliminate assembly or synthesis errors. The functional features (3) and (4) may be carried out in multiple steps and be interleaved with one another.

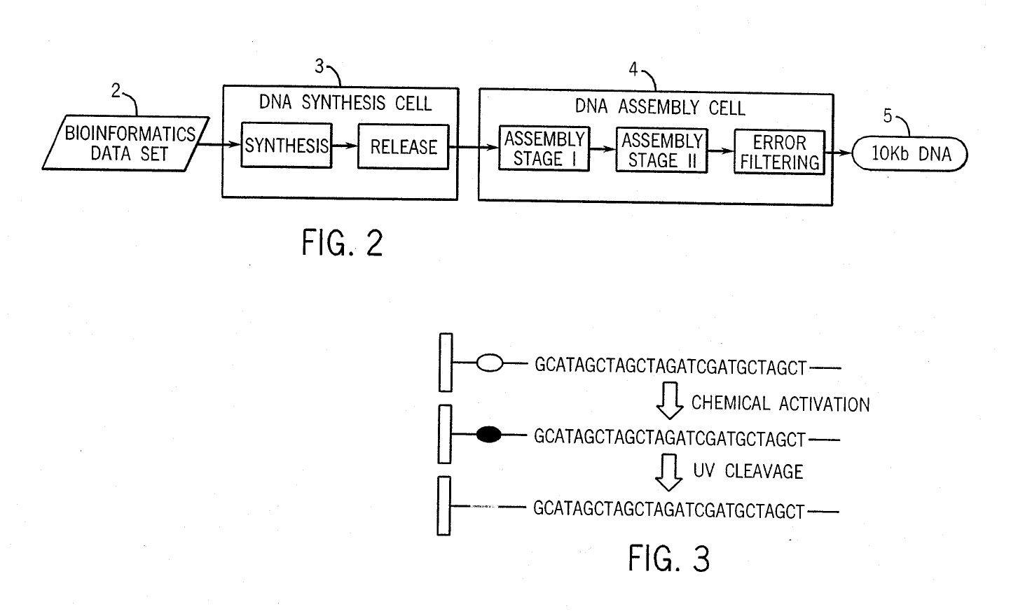

FIG. 2 illustrates the synthesis components. A bioinformatics data set 2 (specifying the oligonucleotides to be synthesized and the assembly sequence, as discussed above) is provided to an automated DNA synthesis cell 3 which carries out oligonucleotide synthesis and selected release of the oligonucleotides, preferably under automated computer control. These materials are then provided to a DNA assembly cell 4 that carries out the assembly stages and error filtering to result in the final synthesized target DNA molecule 5.

The synthesis of oligonucleotides traditionally occurs in the 3′-5′ direction for optimal synthesis yields. For the purpose of creating oligonucleotide microarrays useful in bioassays requiring enzymatic processing of the 3′ ends of the DNA, synthesis in the 5′-3′ direction is required. The quality of oligonucleotides synthesized by inverse 5′-3′ chemistry has been shown to be comparable to that obtained in the normal 3′-5′ direction. Oligonucleotides may be synthesized in either or both directions as needed. For the purposes of gene synthesis, the oligonucleotides need to be released from the support surface, and thus a cleavable linker is required. Standard oligonucleotide synthesis on controlled pore glass substrates utilize a base-labile linker that is cleaved along with the nucleobase protecting groups by ammonium hydroxide or ethylene diamine at the end of the synthesis. Although the base-labile linker approach should be sufficient for the release of oligonucleotides from the glass surface, it requires additional features: (1) the chip surface reactions must be divided into microchannels for the independent release of two or more groups of oligonucleotides for separate assembly, and (2) the DNA is released along with the nucleobase and phosphate protecting group cleavage products, requiring a purification/buffer exchange before the oligonucleotides can be used for assembly. A safety catch photolabile (SCPL) linker is preferably used to allow both the light-directed synthesis and light mediated surface release of oligonucleotides, as illustrated in FIG. 3. This photolabile linker provides several advantages over direct chemical release strategies: (1) the chip layout will be completely flexible for each synthesis as light will dictate which pixels on the chip surface will be released, (2) the purity of the released oligonucleotides will be increased as oligonucleotides will be selectively released with the highest efficiency from the same areas of the chip where the synthesis occurs and not from areas that receive scattered light such as the 1 μm borders surrounding each pixel, and (3) the linker will allow direct release of oligonucleotides into aqueous buffers following deprotection of the phosphate and nucleobase protecting groups.

The quality of synthetic oligonucleotides is governed by a number of factors including: (1) achieving highest possible yield of photodeprotection to obtain acceptable full length products from a multi-step (e.g., up to 80) linear synthesis, (2) the efficiency of attachment of the bases to the deprotected sites (coupling efficiency), and (3) the amount of damage by excess light energy to the growing oligonucleotide strands. To address these issues, methods may be used to speed up the photoreaction and minimize damage to the growing oligonucleotide chains by shifting the deprotection wavelength from the UV to the visible range and suppressing unwanted side reactions during photodeprotection.

Due to the extremely small quantities of oligonucleotides produced per chip (˜10-20 pmol/cm²) utilizing a maskless array synthesizer, highly sensitive methods are required to analyze the quality of the oligonucleotides. Oligonucleotides produced on the MAS chip's surface have been analyzed by cleaving the silicon tether between the linker and the glass slide through extended treatment with ammonium hydroxide, phosphorylating the released oligonucleotides with ATP-y-³²P, and separating the oligonucleotides on the PAGE denaturing gel to visualize the distribution of oligonucleotide lengths produced and to provide a quantitative assessment of synthesis efficiency. The ladders show that the full length products are being produced as the primary products, but also reveal a ladder of truncates, indicating that purification will be required to isolate full length oligonucleotides from truncates and synthesis by-products.

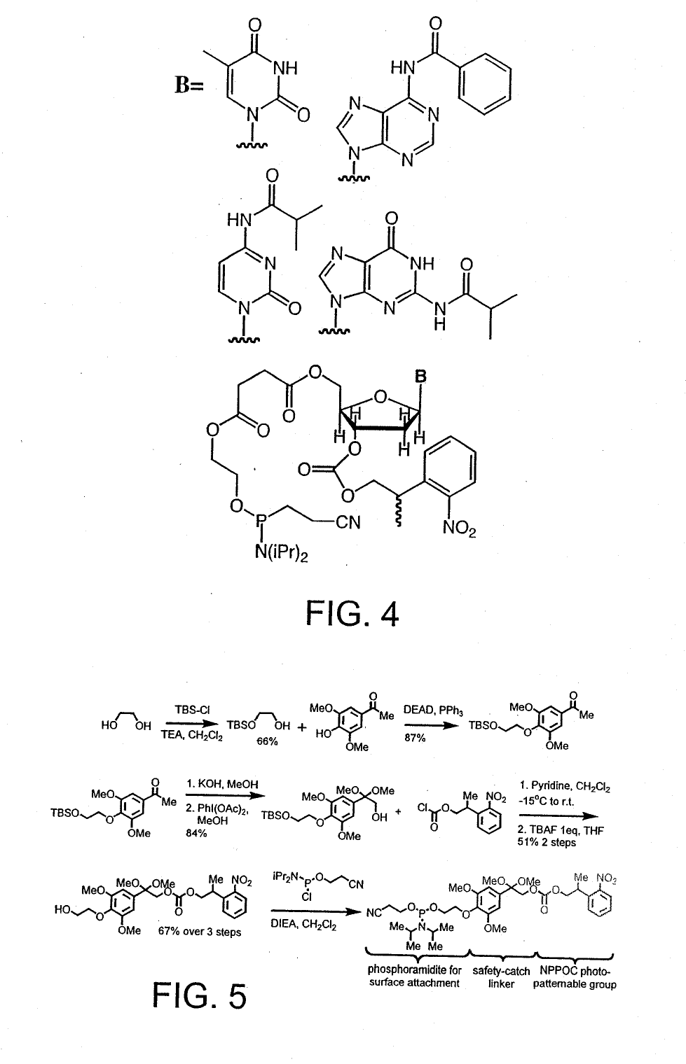

Four examples of specialized photolabile nucleoside phosphoramidites with base-labile linkers are shown in FIG. 4, based upon the acid-labile phosphoramidites described by R. T. Pon, et al., Tetrahedron Lett., 42(51), 2001, p.p. 8943-8946, and may be synthesized as illustrated in FIG. 5. These linkers can be used with 5′-3′ extension phosphoramidites for the optimization of DNA synthesis chemistry.

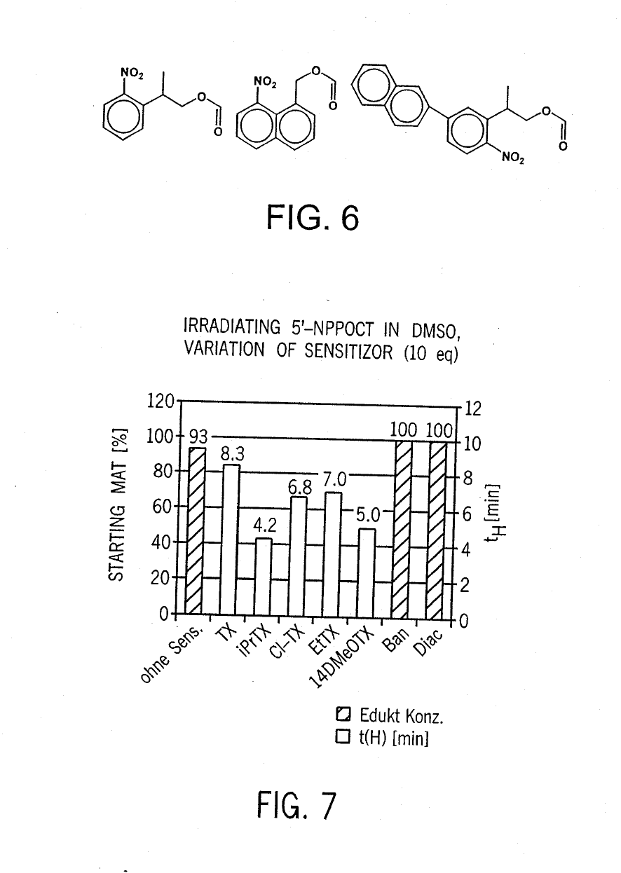

It has been determined that thioxanthone sensitizers increase the quantum efficiency of NPPOC deprotection, that is, the use of sensitizers generates more “light-activated” molecules per photon. New photolabile groups have been developed with faster deprotection rates, improving the speed of photocleavage by about a factor of three. FIG. 6 shows structures of some new light-sensitive protecting groups and their relative deprotection rates (in parentheses). Sensitization of these groups with thioxanthones further enhance deprotection rates by another factor of three; however, the quality of the synthesized oligonucleotides is not optimal due to increased side reactions with the sensitizer chemistry.

Experiments clearly indicate that sensitized deprotection is a viable option for shifting the irradiation wavelength into the visible (>400 nm) region. This is due to the fact that energy band gap between the relevant excited states is smaller in the sensitizer than in the NPPOC. Thus, the necessary wavelength for “populating” the deprotection transition state, the NPPOC-triplet (T1), is shifted from 365 nm to about 405 nm via indirect excitation. As can be seen in the graph of FIG. 7, only a few of the chosen sensitizer molecules effectively deprotect NPPOC-Thymidine at irradiation wavelengths longer than 400 nm.

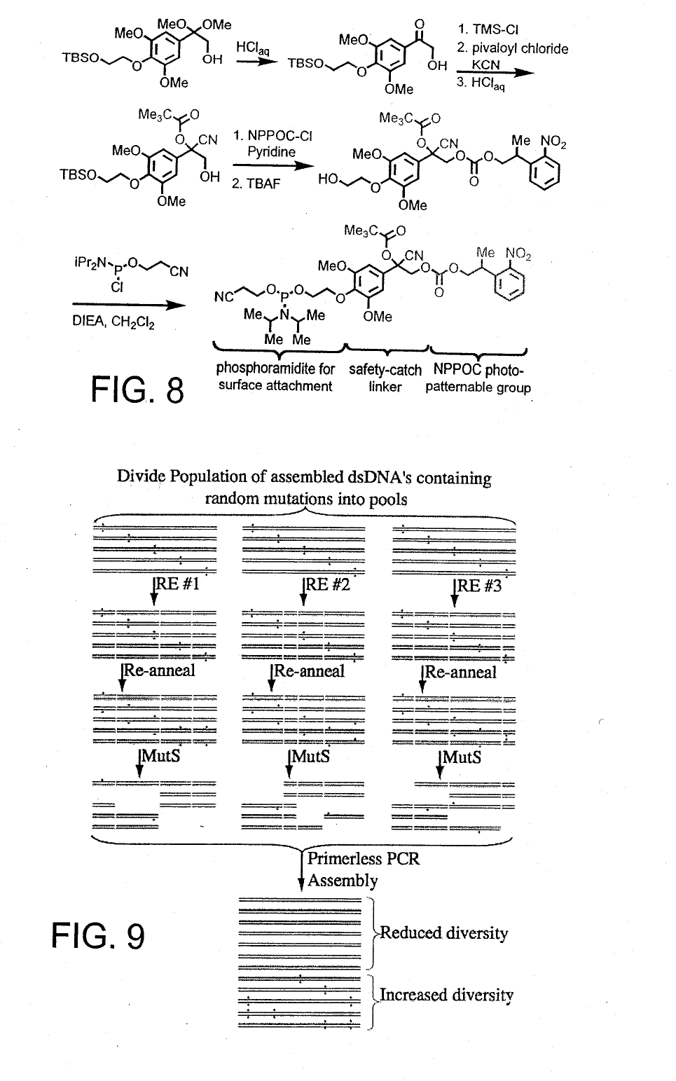

To improve the quality of released oligonucleotides prior to assembly, a reverse phase C18 purification step may be implemented to isolate oligonucleotides that received a base in the final synthesis cycle from those that did not. This should separate primarily full length oligonucleotides from tuncated sequences. In the final cycle, standard dimethoxytrityl (DMT)-protected nucleoside phosphoramidites may be used in place of the NPPOC—protected phosphoramidites such that, after deprotection of the nucleobase/phosphate protecting groups and activation of the safety-catch, oligonucleotides containing a DMT group will be selectively retained on C18-silica. After cleavage of the DMT group with aqueous acid, primarily full length oligonucleotides will be eluted for use in assembly reactions. This trityl-on synthesis and C18 purification is a standard protocol in oligonucleotide synthesis. If this purification is insufficient for assembly, full length oligonucleotides may be isolated by electrophoresis and/or ion exchange chromatography prior to assembly. If separation by oligonucleotide length is required, the oligonucleotide design may be restricted to have all oligonucleotides used in an assembly reaction be of the same length. Where a C18 purification step may be required to remove truncates, a base-activated SCPL-linker may be utilized. A synthesis of a base-activated SCPL-linker is discussed further below and illustrated in FIG. 8. The synthetic route is a minor variation of the existing synthetic route, wherein an acyl cyanohydrin is used to protect the aryl ketone rather than the dimethoxy ketal. This SCPL-linker will be activated by treatment with ethylene diamine while simultaneously deprotecting the nucleobase and phosphate protecting groups prior to photo release. The DMT group is known to be stable to these conditions and will thus allow trityl-on C18 purification.

Although the “building block” nucleotides can undergo filtering and subsequent purification to allow for a reduction in error-filled DNAs, the size of the oligonucleotides themselves may play a vital role in assembly success. Since step-wise base addition is not 100% efficient, the longer oligonucleotides are more likely to have errors and truncate species. However, although the longer oligonucleotides have more errors, fewer of these “blocks” are needed for assembly. The size of the “building block” can have a significant effect on the amount of error introduced into the assembled gene.

One approach for gene assembly in accordance with the invention involves a two stage process in which the synthesized oligonucleotides are first eluted and concentrated prior to assemblage into dsDNA. Assembly (the second stage) occurs in two steps: initially, the 20-50 bp short ssDNA are hybridized together and extended into ever-increasing lengths of dsDNA. After denaturation, this cycle is repeated until the oligonucleotides form the full length template. Next the full length template is amplified by PCR using primers directed against sequences present at the 5′ and 3′ ends of the assembled gene. Amplified products may be cloned and sequenced for quality control. However, depending on the use of the product, large sets of unassembled oligonucleotides or the PCR amplified DNA itself may be provided to the end-user, if desired. In this manner, the picomole concentrations of oligonucleotides present on the glass surface are converted into the nanomole and micromole amounts of DNA needed for cloning.

The two stages (elution and assembly) may be done in one step, but there is a predicted risk of creating truncated amplification products since hybridization is occurring at very low total mass concentrations. Another option involves performing the assembly reaction with the 5′ or 3′ oligonucleotides covalently attached to a small domain on the glass surface. The linker attaching this terminal oligonucleotide to the glass may be either chemically or photolytically labile so that the surface-assembled dsDNA molecule can be released into solution and amplified with the addition of micromole amounts of universal primers.

Results with PCR assembled genes have shown that errors in the initial assembly products are commonplace. These errors limit the immediate usefulness of assembled double stranded DNA for all applications requiring perfect DNA sequences, such as gene expression. Indeed, this problem may be very significant with regard to the length of time required to produce any given sequence, since correcting errors is a time consuming process. To address these problems, general approaches to reduce or eliminate errors in assembled DNA sequences are utilized. There are two distinct phases where additions, deletions, and transversion errors are introduced in synthetic DNA: during the oligonucleotide synthesis; and during the assembly processes. During synthesis, errors can occur through unintended photodeprotections by stray photons, incomplete photodeprotection, incomplete couplings, incomplete nucleobase or phosphate backbone deprotections, as well as plethora of other side reactions. During assembly, errors can be introduced via mls-hybridization or mls-incorporation of bases by the polymerase. Most errors will occur randomly, although some may occur systematically and possibly be sequence dependent. The general preferred approach is termed “consensus filtering” as it utilizes DNA shuffling, error removal, and reassembly to convert a population of DNA molecules with random or partial systematic errors to a population of DNA enriched with molecules containing the consensus sequence of the original population. The error removal process utilizes the mismatch binding protein MutS to remove duplexes containing mismatches via affinity capture from a population of dsDNA molecules. The MutS filter may be considered a “coincidence filter”. The term “coincidence filter” is similar in concept to an “AND” gate in electronic circuitry wherein signal 1 AND signal 2 must be present for an event to be counted. The adaptation of this concept for DNA error filtering works as follows: for every oligonucleotide synthesized on the chip surface, its complement oligonucleotide will also be synthesized. Because the vast majority of the oligonucleotides are wild type (wt) or error-free, the error-containing or mutant type (mt) oligonucleotides will be most likely to hybridize with wild type, thus creating double-stranded oligonucleotides containing mismatches. The mismatched bases in the double-stranded oligonucleotide cause a bulge at the position where the base pairing is incorrect and will thus be trapped by an immobilized MutS protein while error-free pairs will flow through. To ascertain the effectiveness of MutS filtering, a 160 bp region of the green florescent protein (GFP) gene was assembled from unpurified 40mer oligonucleotides. The assembly product was either directly cloned into an expression vector, or heat denatured, re-annealed and subjected to MutS filtering before cloning. Although there were no apparent differences at the functional level (as assayed by visual inspection of the GFP fluorescing transformants), sequence analysis revealed that the control population lacking the MutS filter was 81% wt, whereas the “filtered” population was 100% wt. This experiment demonstrated that MutS filtering can increase the percentage of wt clones. From these and other assembly reactions using PCR, overall mutation rates are between 0.2 and 1.2 errors/kilobase (data not shown). Consensus filtering is essentially equivalent to DNA shuffling with a MutS mismatch removal step. The pool of dsDNA molecules containing mutations is fragmented into sets of overlapping fragments via restriction digestion and re-assembled into full length molecules by primerless PCR and amplification PCR. Although DNA shuffling has traditionally been used as a method for creating diverse populations of DNA molecules with all possible combinations of mutations present in the original population, the creation of diversity from a fixed population of mutants also demands an equivalent reduction in diversity among the shuffled products. Indeed, with this approach it is possible to start with a population of DNA molecules wherein every individual in the population contains errors, and create a new population of molecules in which the dominant species have the consensus sequence of the original population.

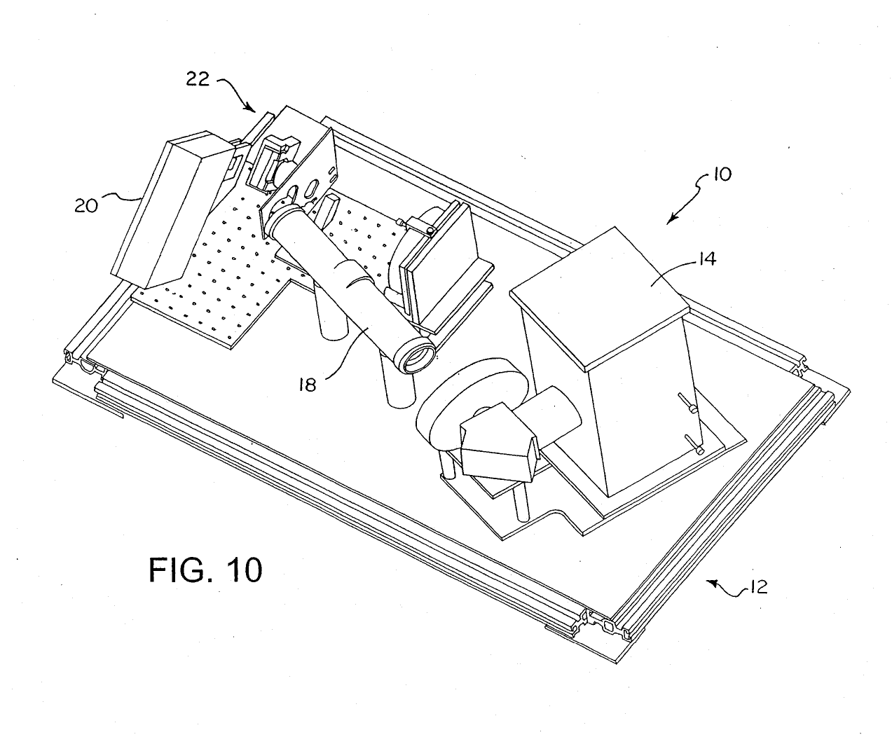

As illustrated in FIG. 9, an assembly PCR product can be split into several pools. Each pool undergoes complete digestion with one or more restriction enzymes to form distinct pools of fragments with overlapping ends. The digested pools of DNA are denatured and re-annealed to create a population of dsDNA fragments wherein the majority of DNA strands containing errors will be present as dsDNAs with mismatches to another strand. This population of DNAs is passed through a MutS filter (MutS immobilized on a solid support) to affinity-remove sequences containing errors. Perfectly matched duplex DNA should pass directly through the MutS filter. The mixture of fragments thus depleted of error containing sequences will serve as template fragments for another assembly reaction. This process can be iterated until the consensus sequence emerges as the dominant species in the population of full length DNA molecules. Implementing shuffling via restriction digests, rather than random fragmentation with DNAse, allows for greater efficiency in MutS filtering by providing double stranded fragments.

The following simple mathematical model can be used to predict some parameters of consensus shuffling.

$$P = 100 {( 1 - \frac{S \cdot E \cdot {M}^{C}}{1000} )}^{\frac{2 N}{S}}$$

Where
P=percentage of clones with no errors
S=average size of fragments
E=errors per 1000 bases of input DNA population
M=MutS factor (fraction of mismatches escaping filter)
C=cycles of MutS filter

An input population of dsDNA molecules of length N, containing E errors/kb is fragmented into shorter dsDNA fragments of average length S. The fraction of oligonucleotide fragments with correct sequences (on average) will be 1−S*E/1000. The likelihood of the assembled product also containing the correct sequence will be the product of the likelihoods of all the individual oligonucleotides used in the assembly having the correct sequence. A reasonable approximation for the required number of oligonucleotides of average length S to assemble a gene of length N is 2N/S, assuming both strands must be represented. If a MutS error filter is applied to the re-annealed dsDNA fragments, the fraction of error containing dsDNA hybrids will be reduced by fraction M, the MutS factor. If the MutS process is iterated to increase the population of correct sequences, the fraction of error-containing sequences (S*E/1000) can be multiplied by the MutS factor M each cycle.

Several interesting predictions emerge from this model. First, some realistic assumptions are made about the variables in this model: error rates in the initial assembly product are between 1 and 5 errors/kb, target sequence lengths are between 500 bases and 5 kb, average fragment lengths are between 50 and 200 bases, MutS factors of 1.0 (no filtering), 0.5 (50% efficient), 0.25 (75% efficient) or 0.1 (90% efficient) are considered. From the results of the theoretical calculations shown in Table 1 below, less than 3 rounds of consensus shuffling with a MutS filter should be sufficient to convert a population of DNA sequences where all molecules contain multiple errors in to a population of DNA sequences where the correct sequence is the dominant sequence. The model also predicts that fragment sizes between 50 and 200 will not be a critical factor, and that MutS filtering, even if poorly efficient (50%) is effective upon multiple iterations.

TABLE 1

| | | | | | Fraction of | % Correct | % Correct | % Correct |
|---|---|---|---|---|---|---|---|---|
| Fragment | Errors | Target | MutS | Oligos per | Incorrect | Consensus | Consensus | Consensu |
| Size | per kb | Length | Factor | Assembly | Fragments | Shuffle (1) | Shuffle (2) | Shuffle (3) |
| S | E | N | M | 2N/S | S*E/1000 | P (C = 1) | P (C = 2) | P (C = 3) |
| 50 | 1 | 500 | 1.00 | 20 | 0.05 | 35.85 | NA | NA |
| 50 | 5 | 500 | 1.00 | 20 | 0.25 | 0.32 | NA | NA |
| 50 | 1 | 5000 | 1.00 | 200 | 0.05 | 0.00 | NA | NA |
| 50 | 5 | 5000 | 1.00 | 200 | 0.25 | 0.00 | NA | NA |
| 50 | 1 | 500 | 0.50 | 20 | 0.05 | 60.27 | 77.76 | 88.22 |
| 50 | 5 | 500 | 0.50 | 20 | 0.25 | 6.92 | 27.51 | 52.99 |
| 50 | 1 | 5000 | 0.50 | 200 | 0.05 | 0.63 | 8.08 | 28.54 |
| 50 | 5 | 5000 | 0.50 | 200 | 0.25 | 0.00 | 0.00 | 0.17 |
| 50 | 1 | 500 | 0.25 | 20 | 0.05 | 77.76 | 93.93 | 98.45 |
| 50 | 5 | 500 | 0.25 | 20 | 0.25 | 27.51 | 72.98 | 92.47 |
| 50 | 1 | 5000 | 0.25 | 200 | 0.05 | 8.08 | 53.47 | 85.53 |
| 50 | 5 | 5000 | 0.25 | 200 | 0.25 | 0.00 | 4.29 | 45.71 |
| 50 | 1 | 500 | 0.10 | 20 | 0.05 | 90.46 | 99.00 | 99.90 |
| 50 | 5 | 500 | 0.10 | 20 | 0.25 | 60.27 | 95.12 | 99.50 |
| 50 | 1 | 5000 | 0.10 | 200 | 0.05 | 36.70 | 90.48 | 99.00 |
| 50 | 5 | 5000 | 0.10 | 200 | 0.25 | 0.63 | 60.62 | 95.12 |
| 200 | 1 | 500 | 1.00 | 5 | 0.20 | 32.77 | NA | NA |
| 200 | 5 | 500 | 1.00 | 5 | 1.00 | 0.00 | NA | NA |
| 200 | 1 | 5000 | 1.00 | 50 | 0.20 | 0.00 | NA | NA |
| 200 | 5 | 5000 | 1.00 | 50 | 1.00 | 0.00 | NA | NA |
| 200 | 1 | 500 | 0.50 | 5 | 0.20 | 59.05 | 77.38 | 88.11 |
| 200 | 5 | 500 | 0.50 | 5 | 1.00 | 3.13 | 23.73 | 51.29 |
| 200 | 1 | 5000 | 0.50 | 50 | 0.20 | 0.52 | 7.69 | 28.20 |
| 200 | 5 | 5000 | 0.50 | 50 | 1.00 | 0.00 | 0.00 | 0.13 |
| 200 | 1 | 500 | 0.25 | 5 | 0.20 | 77.38 | 93.90 | 98.45 |
| 200 | 5 | 500 | 0.25 | 5 | 1.00 | 23.73 | 72.42 | 92.43 |
| 200 | 1 | 5000 | 0.25 | 50 | 0.20 | 7.69 | 53.32 | 85.51 |
| 200 | 5 | 5000 | 0.25 | 50 | 1.00 | 0.00 | 3.97 | 45.50 |
| 200 | 1 | 500 | 0.10 | 5 | 0.20 | 90.39 | 99.00 | 99.90 |
| 200 | 5 | 500 | 0.10 | 5 | 1.00 | 59.05 | 95.10 | 99.50 |
| 200 | 1 | 5000 | 0.10 | 50 | 0.20 | 36.42 | 90.47 | 99.00 |
| 200 | 5 | 5000 | 0.10 | 50 | 1.00 | 0.52 | 60.50 | 95.12 |

Consensus shuffling will be necessary whenever a significant portion of the DNA population contains errors. By fragmenting the full length DNA into shorter fragments, the MutS filter will be able to remove the mismatched fragments while allowing a much greater proportion of the DNA to pass through the filter. In the case where all members of the population contain errors, coincidence filtering of the product alone would be ineffective.

Gene sequence fidelity and production efficiency depend on specificity and completeness of sub-sequence hybridization. The primary bioinformatics objectives are to ensure that each assembly sub-sequence has one and only one complementary target sequence and to ensure that each component sequence is free of any secondary structure that would preclude gene assembly. Thus, the problem of breaking down a complete gene (2,000-10,000 base pairs) into assembly sequences is solved when each of the sequences is unique and structure free.

Bioinformatics software may be utilized to divide a target DNA sequence into oligonucleotides capable of assembly. Effective gene assembly begins with careful planning. The bioinformatics software deconstructs the whole gene into the small oligonucleotide building blocks from which it will be constructed. There are several critical factors that affect the choice of lines of demarcation between assembly sequences. The first step in actual gene assembly is hybridization of sub-sequences. Hybridization between any two indivicial complements should be complete and specific. That means that the thermodynamic stability of the duplex should be known and that the annealing temperature be appropriate to that value. When a sub-sequence has strong secondary structure it cannot effectively hybridize to its complement. Therefore, the potential for secondary structure must be evaluated for each elementary sequence. Next, the potential for mishybridization must be evaluated by identifying gene sequences with a high level of homology to the sub-sequence under consideration. With a fixed annealing temperature, it is possible to predict the extent of mishybridization by calculating the thermodynamic free energy of formation between the sub-sequence and the sequence at the improper target location. The levels of tolerance for secondary structure and mishybridization are difficult to predict without supporting experimental validation.

A relatively simple gene assembly design software breaks the complete gene down into fixed length (N) oligonucleotides. The length is typically 20-60 bases. The length of the overlap between sub-sequences is set at N/2. To find the “best” set of oligonucleotides for assembly, the algorithm divides the sequence into all possible N-mers with N/2 overlap and then calculates the Tm (Tm=81.5+0.41(% GC)−500/length+16.6 log[salt]) of all overlapping portions. The highest score is given to the set with the most uniform set of melting temperatures. The algorithm also scans each overlap sequence for complete uniqueness for its identified target within the context of the entire gene. If more than one target is identified for a sub-sequence, assembly is split to separate the intended target from the unintended target into separate subassembly steps. Sub-assemblies are completed and then combined for the final assembly. Sets with only a few sub-assembly steps are scored more favorably than those with multiple assembly steps. The output of the software is the set of oligonucleotides with the best overall score. In a more sophisticated software approach, the gene is still divided into fixed length (N) sub-sequences, but instead of simply having fixed N/2 overlaps, overlap length is adjusted to achieve a specific melting temperature (% G/C method).

The software may have a web based graphical user interface based on the design of the familiar NCBI BLAST interface. The user can paste or upload a sequence file of the desired DNA sequence into the sequence window. The user then chooses the sub-sequence length and the desired assembly temperature. The user can also specify the coordinates of the open reading frame and choose from a menu of codon preferences for the output oligonucleotides. This feature enables sequences from one species to be efficiently expressed in another. The output is displayed in two formats. The text mode displays lists of oligonucleotides with their melting temperatures broken up into assembly steps. The graphics mode visually shows the oligonucleotides and overlaps. Each image of a fragment is a link to a text string representation of that fragment sequence. The two modes have clickable links to an output tab delimited file containing the list of oligo sequences to be synthesized, its step, and its overlap melting temperature. The links allow the user to open or save the file.

Various adjustments and enhancements may be made to the basic software structure. A first adjustment updates the method of calculating melting temperature to one that uses nearest neighbor (NN) free energies. The accuracy of the NN method is significantly higher than the % GC method. A second adjustment eliminates the requirement for fixed length product. Rather, an assembly Tm can be defined and the length of sub-sequence products adjusted in each case to be the sum of two variable length sequences chosen to agree with the design Tm. Once the entire gene is broken down into parts, each part can be evaluated for secondary structure (e.g., hairpin information) using the publicly available Mfold or other similar software packages. Such programs have been used to evaluate large combinatorial libraries (17 million individual sequences) of long 100mer oligonucleotides for secondary structure and cross-hybridization between individual members. Sets for the synthesizer can be scored highly which have little or no secondary structure at the assembly temperature. The overlapping sequences are tested for uniqueness in the gene and near-identical sequences can be evaluated as potential sources of error. Specifically, partial match sequences can be identified which may contain mismatches, insertions, or deletions, and their thermodynamic binding energy can be calculated. The error prone sequences (those whose free energies indicate unacceptable levels of formation at the design Tm) can either be separated during assembly or an alternate set will be chosen which divides the conflicting sequences. Finally, the software can automatically perform a BLAST search for each gene sequence to ensure that it does not contain significant sub-sequences of forbidden pathogens (Anthrax, Plague, Ebola etc.)

There are four critical aspects of the multiplexed surface invasive cleavage reaction bioinformatics that deserve attention. First, one must consider the uniqueness of each probe and its specificity for the desired target in the context of the complete sample. While it is quite straightforward to ensure that the complete probe sequence is unique, one also must consider non-specific hybridization, which would inhibit proper signal generation. Second, one must consider the uniformity of duplex formation temperature. For the invasive cleavage reaction, the optimum reaction temperature is identical to the melting temperature of the target:probe duplex. Duplexes whose formation temperatures differ from the reaction temperature may not produce large signals because of limited cleavage. Third, it is becoming well known that the duplex formation energies are lower on surfaces than in solution. The reasons are just now being elucidated. This fact must be accounted for when choosing sequences and reaction temperatures. Fourth, in one of its current forms, the surface invasive cleavage reaction requires addition of invader oligonucleotides in solution. It is important that these oligonucleotides also have high specificity for the target and additionally do not hybridize to any probes at the reaction temperature. This concern is obviously eliminated for the second format of the reaction where both invader and probe are co-immobilized on the same array element.

After the set of oligonucleotides has been selected, synthesis of these oligonucleotides is preferably carried out utilizing an automated DNA synthesizer system. Because of its flexibility and addressability, a large massively parallel optical DNA maskless array synthesizer (MAS) system which is based on the use of a high density spatial light modulator (e.g., as described in U.S. Pat. No. 6,375,903, incorporated herein by reference) is a preferred system for oligonucleotide synthesis. An image locking system as described below is preferably used to eliminate image drift during synthesis of the set of oligonucleotides.

FIG. 10 illustrates a schematic of an optical system 10 of an MAS gene synthesizer incorporating image locking. The system 10 includes a 1:1 ratio image projection system 12, a mercury (Hg) arc lamp 14, an image locking system 16, a condenser 18, a digital micro-mirror device (DMD) 20, and a DNA cell 22. The digital micromirror device (DMD) 20 may consist of a 1024×768 array of 16 μm wide micro-mirrors. Preferably, these mirrors are individually addressable and can be used to create any given pattern or image in a broad range of wavelengths. Each virtual mask is generated in a bitmap format by a computer and is sent to the DMD controller, which forms the image onto the DMD 20. The 1:1 ratio projection system 12 forms a UV image of the virtual mask on the active surface of the glass substrate mounted in a flow cell reaction cell connected to a DNA synthesizer.

A maskless array synthesizer can generate several μm of drift over several hours due to the thermal expansion of optics parts and from other sources. The optical path between the DMD 20 and DNA cell 22 is about 1 meter. The thermal expansion caused by the temperature and humidity fluctuation of surrounding environments and also due to UV exposure, a slight change of position or rotation of the primary spherical mirror and other optical parts may result. This slight change may cause several μm of drift of the projected image. Since the space between each digital micromirror is only 1 μm, this image drift can cause the projected image to be shifted to expose the UV light at the wrong oligonucleotide spots, generating defects in oligonucleotides sequences and their spatial distribution. The image locking system 16 confines the image shift within a certain range to minimize image drift.

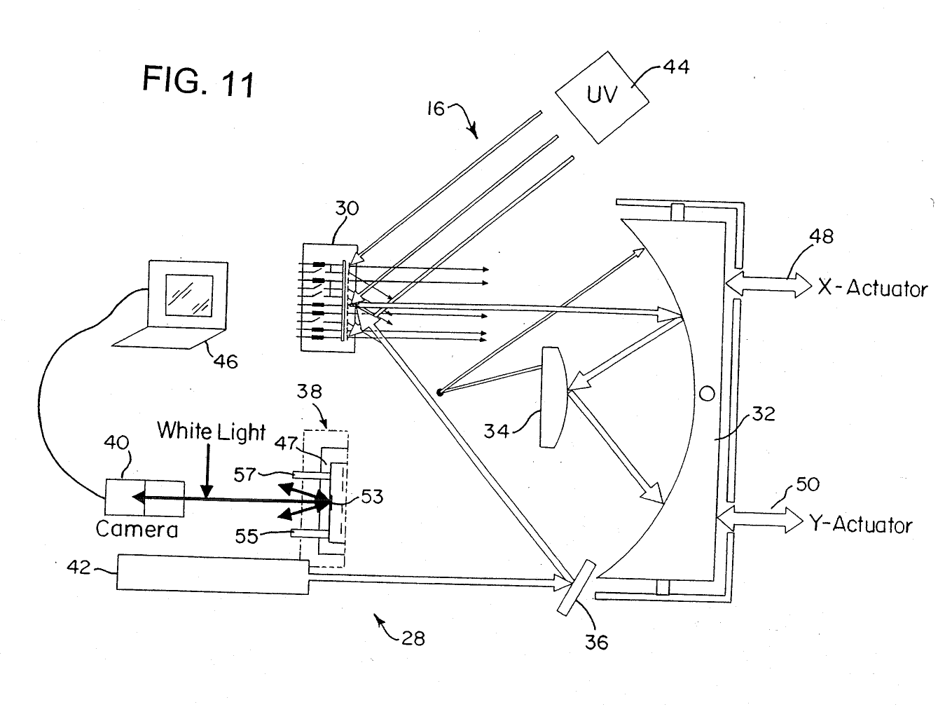

FIG. 11 illustrates a diagram of an image locking system 28. The image locking system 28 can include a digital light processor (DLP) or digital micromirror device (DMD) 30, a concave mirror 32, a convex mirror 34, a beam splitter 36, a reaction cell 38, a camera 40, a laser 42, and a UV lamp 44. In an exemplary embodiment, the laser 42 is a He—Ne laser with a wavelength of 632.8 nm (red light) and does not disturb the photochemical reaction of oligonucleotide synthesis. The He—Ne laser beam from the laser 42 is projected to a reaction cell 38 using an “off” state (rotated −10°) of micromirrors without interrupting the current UV exposure system with UV light from the UV lamp 44 which is projected to the reaction cell 38 using an “on” state (rotated 10°) of micromirrors. The He—Ne laser 42 is at the opposite side of the UV lamp 44 with incident angle of −20° into the DMD 32.

The system 28 can be a 0.08 numerical aperture reflective imaging system based on a variation of the 1:1 Offner relay. Such reflective optical systems are described in A. Offner, “New Concepts in Projection Mask Aligners,” Optical Engineering, Vol. 14, pp. 130-132 (1975). The DMD 30 can be a micromirror array available from Texas Instruments, Inc. The reaction cell 38 includes a quartz block 47, a glass slide 49, a projected image 51, a radiochromic film 52, and a reference mark 53. The UV lamp 44 can be a 1000 W Hg Arc lamp (e.g., Oriel 6287, 66021), which can provide a UV line at 365 nm (or anywhere in a range of 350 to 450 nm). Other sources, such as, e.g., Ar-ion lazers and Hg—Xe high pressure lamps, may also be used.

The laser 42 projects a laser beam onto beam splitter 36 which reflects a portion of the beam onto DMD 30. DMD 30 has a two-dimensional array of individual micromirrors which are responsive to the control signals supplied to the DMD 30 to tilt in one of at least two directions. A telecentric aperture may be placed in front of the convex mirror 34.

The camera 40 is a closed circuit device (CCD) camera used to capture an image of one or more alignment marks. The captured image is transferred to a computer 46 for image processing. When a misalignment is detected, correction signals are generated by the computer 46 and sent to actuators 48 and 50 as the feedback to adjust the mirror 32, so that the correct alignment is reestablished. In at least one alternative embodiment, three electro-strictive actuators (instead of actuators 48 and 50) are used to provide minimum incremental movement of 60 nm and control the rotations and movement of the mirror 32. The displacement of the projected image at the glass slide is highly sensitive to the rotations and movement of the mirror 32.

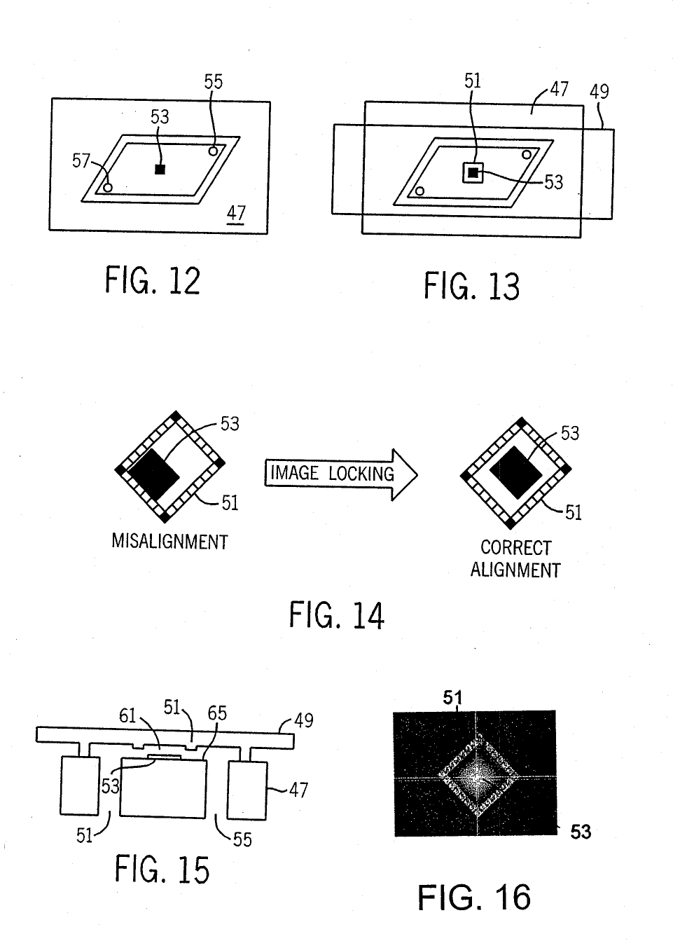

FIG. 12 illustrates the alignment mark 53 patterned on the quartz block 47 in the reaction cell 38. The quartz block 47 includes an outlet 55 and an inlet 57 through which fluid may flow through the reaction cell 38. Such reaction cells are described in U.S. Pat. Nos. 6,375,903, 6,315,958, and 6,444,175. A predefined micromirror pattern shown in FIG. 13 is projected, being centered at the alignment mark 53. In an exemplary embodiment, the projected image 51 is manually aligned at the beginning of synthesis, so that the center of the projected image 51 is overlapped with the center of the alignment mark 53. The CCD camera 40 is used to capture the image that is formed by a 20× (long focal length) microscope lens, which is focused at the middle between the reference mark 53 and the projected image 51. An image processing program in the computer 46 calculates the centers of the reference mark 53 and the projected image 51, generating the amount and direction of any displacement, and sending its correction signals to the corresponding actuator(s) 48 and/or 50. The reference mark 53 is patterned on the surface of the quartz block 47 as shown in FIG. 12. The relative position of the projected image 51 to the reference mark 53 is shown in FIG. 14.

FIG. 15 illustrates a cross-sectional view of the reaction cell 38. The projected image 51 is focused on an inner glass slide surface 61 of the glass slide 49 where the oligonucleotides are grown. The reference mark 53 and the projected image 51 are not at the same focus plane. A microscope lens focuses at the middle plane between the reference mark 53 and the projected image 51. As such, the image captured by the camera 40 is blurred, as shown in FIG. 16. The gap between the glass slide surface 61 and quartz block surface 65 of the quartz block 47 is on the order of 100 μm. To locate the center position of each pattern, a 2D optical pattern recognition technique, which is based on correlation theory, is used. Correlation analysis compares two signals (or images) in order to determine the degree of similarity, where input signal is to be searched for a reference signal. Each correlation gives a peak value where the reference signal and input signal matches the best. If the location of this value is different from the previous value, it means that the image has been shifted, indicating the need of correction.

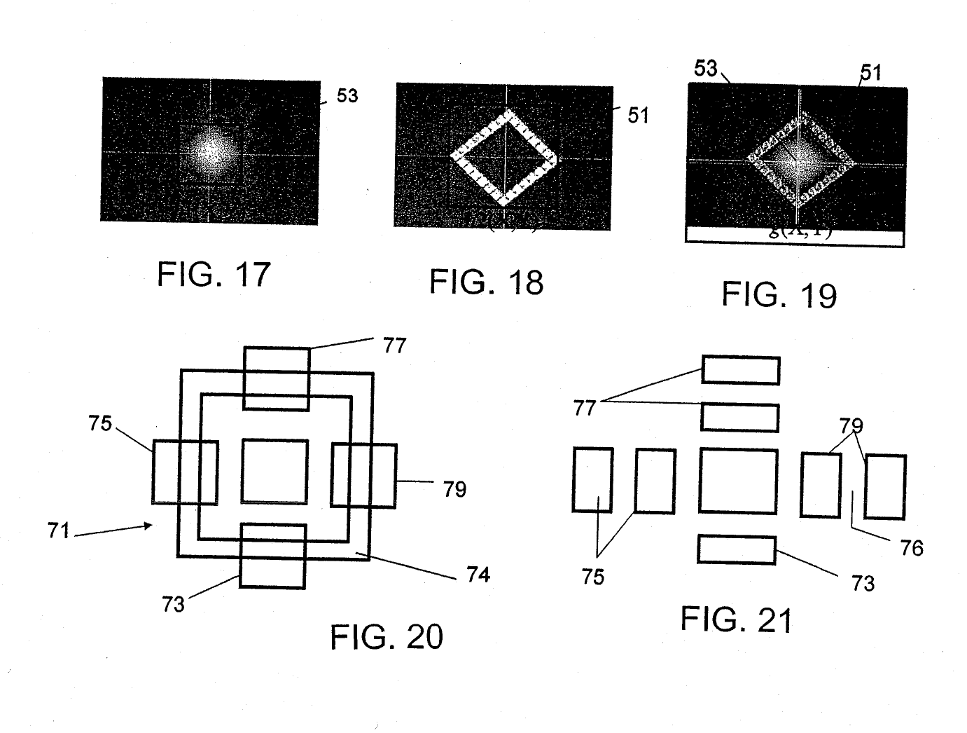

In an exemplary embodiment, an image processing procedure calculates the image displacement from the images captured by the camera 40, by calculating the cross-correction signals between a captured input image described with reference to FIG. 19, the reference mark 53 of FIG. 17, and the projected image 51 of FIG. 18. The cross-correlation is a measure of the similarity between two images, such as images from FIGS. 17 and 19 and such as images from FIGS. 18 and 19. Mathematically, the cross-correlation can be calculated as:

$${c}_{gh} ( X , Y ) = {\int }_{- ∞}^{∞} {\int }_{- ∞}^{∞} g ( x , y ) h ( x + X , y + Y ) ⅆ x ⅆ y$$

or, using the Wiener-Khintchine Theorem, as
c_{gh}(X,Y)=IFFT(FFT2(g(X,Y))·FFT2(rot90(h(X,Y))))

The new locations of the reference mark and the projected image are marked by correlation peaks (i.e., the highest value of c_{gh}(X,Y)). Based on the new locations, correction signals are computed and sent to the actuators to move the mirror. This correction procedure continues until the synthesis is completed.

In an exemplary embodiment, computer programs control the actuators and generate the correction signals by image processing. A log file of displacements can also be recorded and analyzed for measuring actual displacement indirectly and its direction for further refinement of the algorithm. Various mark shapes (e.g., crosses, chevrons, circles) can be used as the reference mark 53.

FIG. 20 illustrates an image 71 projected on a substrate where the image includes several micro-mirrors 73, 75, 77, and 79 according to another exemplary embodiment. A reference mark 71 is included on the substrate. In the field of the microscope, the micro-mirrors 73, 75, 77, and 79 appear as a bright image while the reference mark 74 can be dark so that the image of the mask will appear as a dark line 76 (FIG. 21). As such, overlap of the micro-mirrors 73, 75, 77, and 79 and the reference mark 74 can be observed. Image processing software can determine if the dark shadows are centered on the micro-mirror and if not, apply a correction.

Since each pixel is approximately 15 μm in size, it is necessary to keep the image locked to less than 200 nm. Since the distance from the concave mirror 32 (FIG. 11) to the reaction cell 38 can be approximately 500 mm, the angle pointing accuracy is 0.4×10⁻⁶ radians. Since the diameter of the optics is 200 mm, a piezoelectric or similar system can be used to generate the angular shift by applying a displacement of 80 nm. Typically, a nanopositioner can control displacements of even 10 nm. In particular, the focus of the system can be adjusted by moving the three actuators together (piston motion). The focal position is affected by the distance between the fixed small mirror and the movable large mirror.

Other designs are possible, involving different schemes for the detection of the displacements. The actuators 48 and 50 can be used to effectively align the optics. In another exemplary embodiment, diffractive marks can also be used, alleviating the need for microscopes. Partially transmitting marks (half toned) can be used for other schemes of detection.

The synthesis stage may utilize the technology that has been developed for the fabrication of rapid turnaround microarray DNA chips and that is being commercialized by NimbleGen, Inc. See, e.g., F. Cerrina, et al., Microelectronic Engineering, 61-2, 2002, pp. 33-40. In this process, oligonucleotides are attached to the substrate by a stable linker, and are terminated with a photolabile protecting group. Exposure to the light removes the photolabile protective group, making the attachment point available to chemicals that are floated into the reaction cell. These chemicals can be phosphoramidite based, or can be other types of more general chemicals, and carry the photoprotecting group. After attachment of the base (the chemicals to be attached will be referenced to as “base” although other molecules are possible), the base is connected to the pre-existing oligonucleotide and the photolabaile group protects it from further development. After four of these steps, one per base, the surface of the chip will have an array of the four different “colors,” i.e., A, C, T or G. In the next round of exposure, the photolabile groups are again deprotected by selective light exposure and the next base is attached. In this way, if N illuminated pixels are used to form the exposure, at the end of 20 cycles N different oligonucleotides will be distributed on the surface of the chip in separate and distinct locations. The areas where the oligonucleotides have been synthesized are “tiled” on the surface and are separated from each other by a region where no exposure takes place. This reduces the problem of light being scattered from one tile into the other and thus into causing unwanted reactions. The use of digital micromirror display (DMD) based optics as discussed above allows great flexibility in the DNA chip layout. To completely deprotect a site requires about 60 seconds at a fluence of about 100 mw/cm² of Hg I-line radiation (365 nm). Throughout the system, great care is used to contain stray and diffracted light because photons that reach unwanted sites will cause unwanted deprotection reactions and thus errors in the synthesis. Stray light must be kept to an absolute minimum. This may be done by using high quality optical mirrors and anti-reflection coatings on all of the surfaces that are present throughout the system.

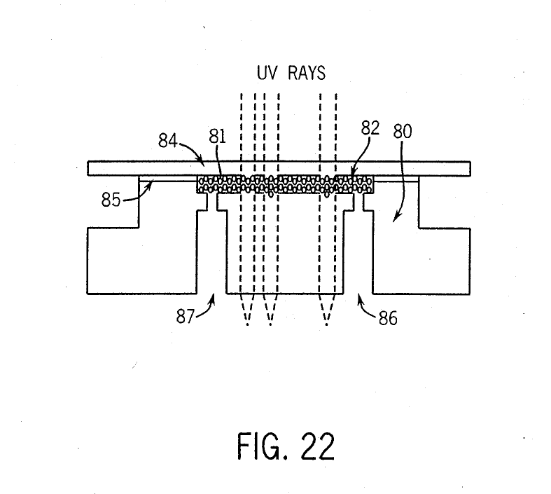

In the formation of the oligonucleotides for gene synthesis, the dimensions of the features are usually relatively large, approximately 100×150 microns. That means that the geometrical depth of focus of the image is of the order of 1400 microns at a NA of 0.07, while the cavity of the typical reaction chamber is only of the order of 100 microns. As shown in FIG. 22, the synthesis chamber of a reaction cell 80 (e.g., formed from a quartz block) can be modified to increase the active surface area by filling the chamber 81 of the cell with quartz microspheres 82 that have been primed before insertion into the chamber. The chamber 81 is defined between a well in the reaction cell block and a glass slide 84, sealed by a gasket 85. A fluid inlet 86 and fluid outlet 87 allow fluid to be introduced into and removed from the chamber. The active surface area is greatly increased by performing the synthesis on the microspheres 82 rather than on the flat surfaces of a glass slide. The spheres cannot move around during the synthesis because of a combination of tight packing and surface tension, and thus do not compromise the quality of the imaging during the synthesis. A liquid index matching fluid can be used during the exposures so that the spheres themselves will be essentially invisible to the incoming light and not affect the image.

Synthesis may also be carried out by other types of systems, for example, based on the use of an array of light emitting diodes (LEDs) or solid state lasers. Such an array can be placed at the focal plane of the mirrors assembly, replacing the micromirror spatial light modulator and lamp. Several types of LEDs are commercially available, based on gallium nitride and/or aluminum nitride formulation with different lifetimes and different wavelength characteristics, from companies such as Nichia, Cree and Uniroyal. An array of solid state lasers may also be used instead of an array of LEDs.

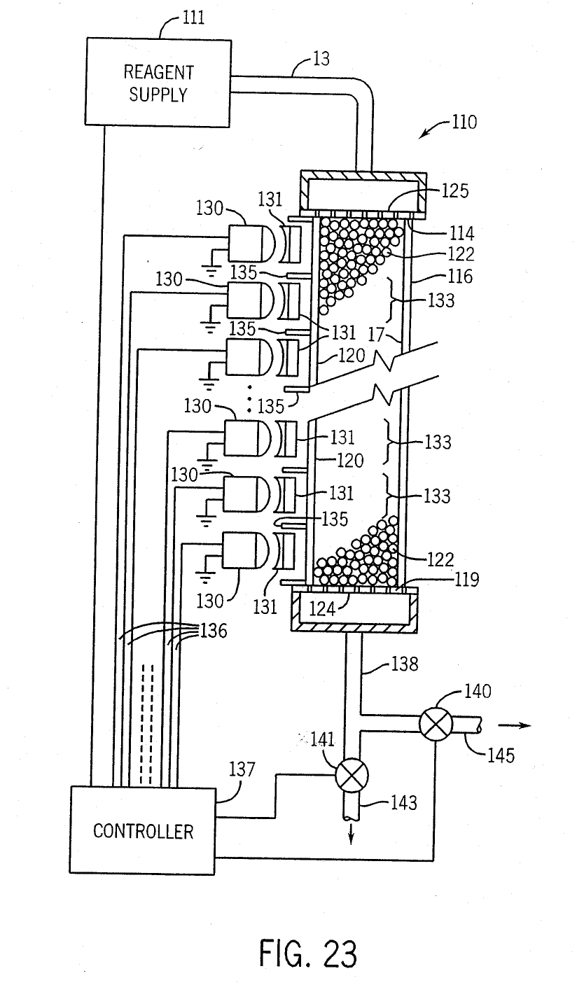

Other types of automated synthesis systems may also be utilized that do not rely on optical image formation to form an array. For example, synthesis can also be carried out utilizing a column packed with microspheres as illustrated in FIG. 23. Such a parallel synthesizer is capable of creating many (e.g., 20) different sequences at once using photolabile chemistry. Several such parallel synthesizers may then be used to release selected nucleotides formed therein to an assembly chamber where assembly of longer DNA fragments takes place. The active area of the microspheres is much larger than the surface area of a glass slide or chip used in forming microarrays. In addition, the spheres occupy part of the volume so that the amount of reagent used need only be an amount sufficient to fill the free volume among the spheres. The net result is that the ratio of synthesis surface area to reagent volume is much greater than in flat surface synthesis.

In the apparatus 110 shown in FIG. 23, a reagent supply 111 is utilized to provide selected reagents, as discussed further below, in sequence on a supply line 113 that provides the liquid reagents to the inlet end 114 of a conduit 116. The conduit 116 has an interior channel 117 through which the reagents flow to an outlet end 119 of the channel in the conduit. The conduit 116 can be formed as a thin walled capillary tube in which the channel 117 is the cylindrical interior bore of the capillary tube conduit. The wall 120 of the conduit 116 may be formed of a substantially transparent material, such as glass or quartz, so that light from outside the conduit can be transmitted through the wall of the conduit and thence into the interior channel 117. The channel 117 holds a large number of solid carrier particles 122 which may be spherical as shown, but which may also have other shapes such as cylinders or fibers, etc., formed of a variety of materials such as quartz, glass, plastic, and, in particular, CPG glasses and other porous materials. The particles 122 may have sections of different sizes or optical properties to better control flow of reagent, improve the exposure uniformity and better control scattered light. The particles 22 may be held within the channel 117 by a perforated screen 124 at the outlet 119 of the channel and preferably also by a screen 125 at the inlet end 114 of the channel. The screens 124 and 125 have openings formed therein which are sized to allow fluid from the reagent supply 111 to pass freely therethrough while blocking passage of the carrier particles 122 through the openings, thus holding the particles 122 within the channel without fixing or attaching the particles to the walls of the channel. The fluid from the reagent supply flows through the interstices between the particles 122 so that the flowing fluid is in contact with a large proportion of the surface area of the particles 122 as the fluid flows through the conduit. Thus, the total area on which chain molecules can be formed is many times greater than the interior surface area of the channel 117, and generally is far greater than the surface area of the flat substrates conventionally used in DNA microarrays. The reagent supply 111 may be, for example, a conventional DNA synthesizer supplied with the requisite chemicals.

A plurality of controllable light sources 130 are mounted at spaced positions along the length of the transparent wall 120 of the conduit to allow selective illumination of separated sections of the conduit and of the particles held therein in the separated sections. Light emitted from the sources 130 may be focused by lenses 131 before passing through the wall 120 of the conduit to illuminate separated sections 133 of the particles within the conduit. Light absorbing or blocking elements 135 may be mounted between each of the light sources 130 to minimize stray light from one light source being directed to the region to be illuminated by an adjacent light source. The light sources 130 may be any convenient light source, for example, light emitting diodes (LEDs), which are selectively supplied with power on lines 136 from a computer controller 137, such that any combination of the light sources can be turned at a particular point in time. Any other controllable light source may be utilized, including individual lamps of any type that can be turned on and off, constantly burning lamps with mechanical shutters (including movable mirrors as well as light blocking shutters) or electronic shutters (e.g., liquid crystal light valves), and fiber optic or other light pipes transmitting light from single or multiple sources, etc. The controller 137 is also connected to controllable valves 140 and 141 which are connected to an output line 138 which receives the fluid from the outlet end 119 of the conduit. The controller 137 can control the valves 140 and 141 to either discharge the reagents that have been passed through the conduit onto a waste (collection) line 143, or to direct oligomers which have been released from the conduit onto a discharge line 145 which can be directed to further processing equipment or to readers, etc.

In operation, the reagent supply initially provides fluid flowing through the conduit that creates a photodeprotective group covering the surfaces of the carrier particles 122. The flow of reagent is then stopped and the controller 137 turns on a selected combination of the light sources 130 (typically at ultraviolet (UV) wavelengths) to illuminate selected ones of the separated sections 133 of the packed particles within the conduit. In a conventional manner, the light emitted from each active source 130 renders the photodeprotective group susceptible to removal by a reagent which is passed through the conduit by the reagent supply 111, following which the reagent supply can be controlled to provide a desired molecular element, such as a nucleotide base (A,G,T,C) which will bind to the surfaces of the carrier particles from which the photodeprotective group has been removed. Thereafter, the reagent supply can then provide further photodeprotective group material through the conduit to protect all bases, followed by activation and illumination from selected sources 130 to allow removal of the photodeprotective group from the particles in selected sections of the conduit. After removal of the susceptible photodeprotective material, the reagent supply 111 can then provide another base material that is flowed through the conduit to attach to existing bases on the carrier particles which have been exposed. The process as described above can be repeated multiple times until a sufficient size of chain molecule is created. Each of the light sources 130 can separately illuminate one of the separated sections of packed particles, allowing different sequences of, e.g., nucleotides within the oligomers formed at each of the separated sections.

Although it is preferable that the controller 137 be an automated controller, for example, under computer control, with the desired sequence of reagents and activated light sources 130 programmed into the controller, it is also apparent and understood that the reagent supply 11 and the light sources 130 can be controlled manually and by analog or digital control equipment which does not require the use of a computer.

The surfaces of the carrier particles 122 are coated with a material that acts as a group linker between the surface of the particle and the chain molecule to be formed. The carrier particles may have a diameter substantially less than the width of the channel so that multiple carrier particles may pack each section of the channel between the walls of the channel. The carrier particles are otherwise free from attachment to each other or to the walls of the conduit. As illustrated in FIG. 23, the conduit may be formed of a thin walled capillary tube and the carrier particles may comprise spherical quartz particles of a diameter from a few microns to several hundred microns or more. However, the conduit may also be formed in other ways, including solid fluid guiding structures, in which the channel is formed within the solid structure of the conduit, and the carrier particles may be formed in shapes other than spheres, for example, as cylinders, fibers, or irregular shapes, and with smooth or structured surfaces. For example, the carrier particles may be formed of controlled porosity glass (CPG) or similar porous materials which provide a large surface area to mass ratio. The particles may be contained in other ways, for example, trapped in wells formed in a substrate, rather than being contained in a tube.

The light sources emit light within a range of a selected wavelength, and lenses and/or mirrors may be mounted with the sources to couple and focus the light from the sources onto the sections of the channel. The sources may also be mounted to the conduit such that a face of the source (e.g., a light emitting diode) from which light is emitted forms a portion of the transparent wall of the conduit. Light blocking material may be mounted between adjacent sources in position to prevent light from one source passing into a section of the channel that is to be illuminated by an adjacent source. The conduit may be filled with an index matching fluid to minimize scattering losses. The apparatus may further include a transparent window spaced from the transparent wall of the conduit and including an enclosure forming an enclosed region with the window and the transparent wall of the conduit. An index matching fluid within the enclosed region has an index of refraction near that of the transparent wall of the conduit to minimize reflections at the transparent wall of the conduit. The light sources may be mounted outside of the window in position to project light through the window, the index matching fluid, and the transparent wall of the conduit. The window can include an antireflective coating thereon to minimize unwanted reflections and dispersion of light. Where the conduit has walls which are all transparent to light, a material may be formed adjacent to the conduit, between the separated sections to be illuminated, which absorbs or reflects light transmitted through the walls of the conduit to minimize stray light.

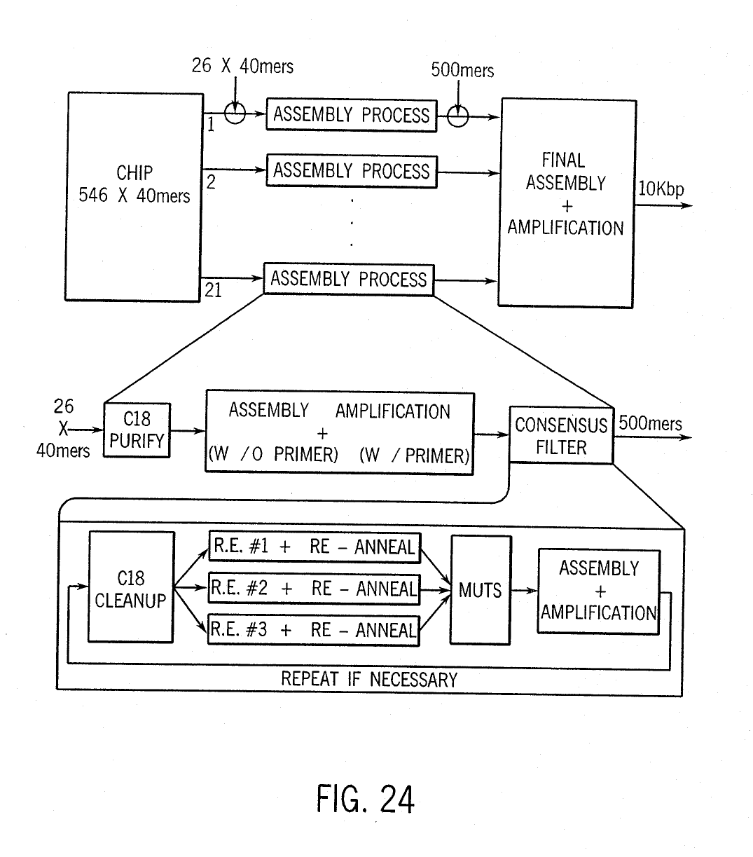

FIG. 24 illustrates an exemplary assembly process in accordance with the invention. This process is shown for illustration as utilizing a “chip” (with a flat support substrate) formed using a maskless array synthesizer, but it is understood that the same process may be carried out with other synthesizers, such as multiple column synthesizers as shown in FIG. 23, which release oligonucleotides in sequence in a manner similar to which oligonucleotides are released from an array formed on a chip. For example, to assemble a 10K bp gene from 40mer oligonucleotides, 549 unique 40mers are synthesized on the DNA chip in a single run. It is understood that not all the oligomers need to be or generally will be of the same length. In this particular example, a group of 26 unique 40mers is eluted from the forming support surface and may then be purified using a reverse phase C18 column to filter out non-full length oligonucleotides from the synthesis product, although other filtering approaches may be used. The purified group of 40mers is assembled to generate an intermediate 500mer, which is then amplified using polymerase chain reaction to increase the concentration. Before assembly of the 21 packs of 500mers into a 10K bp gene, each 500mer may also go through a consensus filter, as discussed above, to remove the errors introduced during assembly via mls-hybridization or mls-incorporation of bases by the polymerase. The pool of 500mer dsDNA molecules containing mutations is fragmented into sets of overlapping fragments via restriction digestion and re-assembled into full length molecules by primerless PCR and amplification PCR. The whole assembly involves several steps performed in a serial manner. After the oligonucleotides are synthesized and eluted, subsequent purification, assembly, PCR, and error-filtering steps may be done manually or automatically.

After synthesis and elution, volumes of materials may be handled through a repetitive process. The post-synthesis steps can be automated using a microtiter plate preparation robotic workstation. In this approach, the oligonucleotide sets are selectively eluted to individual wells in a (e.g., 96-well) microtiter plate. Then, these oligonucleotides are purified using an array of C18 pipette tips mounted on the robotic tool head, as illustrated in FIG. 25. The reverse phase C18 purification requires two steps. First, the desired oligonucleotides with the trityl protecting group are retained in the C18 filter during the “catch” cycle, allowing undesired oligonucleotides and other salts to pass through. Next, during the “release cycle,” the trityl group is cleaved by an acid to release the oligonucleotides to another microtiter plate, which is transported and loaded into a thermal cycler for assembling short ssDNA 40mer oligonucleotides into an intermediate 500mer. The assembly step may be performed in a 96-well titer plate thermal cycler. The C18 purification step requires carefully controlling the fluidic flow to gain maximum yield. Modification to the tool head or control algorithm of the workstation can be utilized to satisfy the accurate flow control requirements.

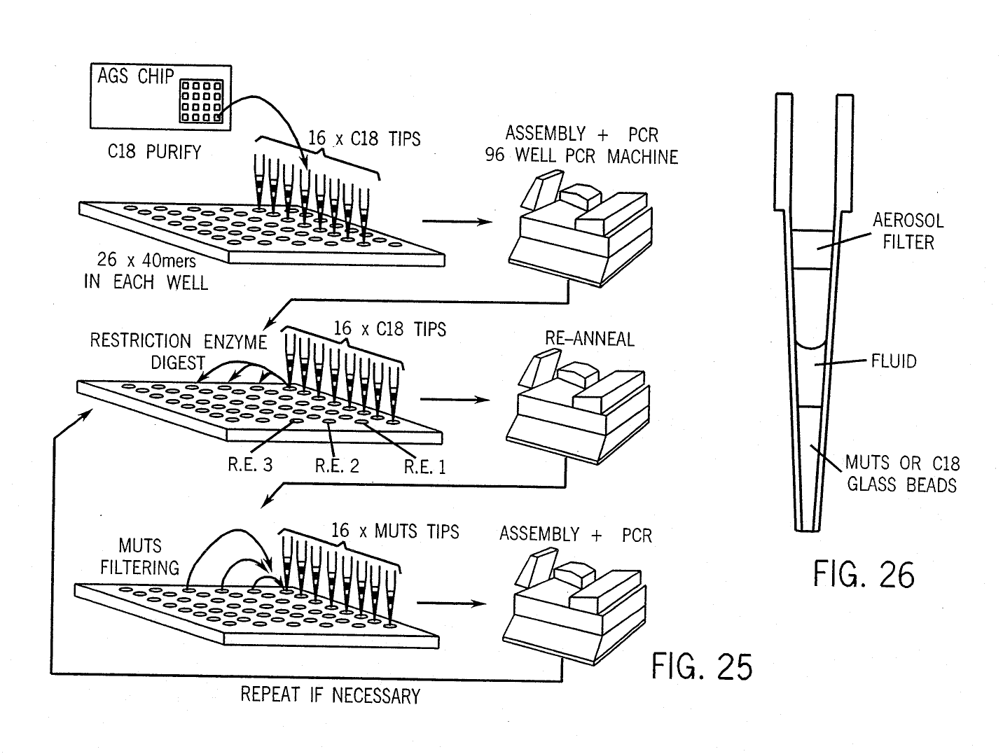

Each assembled 500mer pool is purified using another C18 array to remove the polymerase enzyme and then dispensed into three wells (pools) with equal volume to perform consensus filtering. Each pool undergoes complete digestion with one or more restriction enzymes. The digested pools of DNA are denatured and re-annealed using the cycler. The MutS filtering step can also be accomplished using parallel pipettes and fluid dispensing. The MutS pipette tips may be formed as shown in FIG. 26. The flow velocity for the dispensing step should be tightly controlled. The consensus filtering steps may be repeated if necessary. Once the assembly step is complete, the filtered oligonucleotides are dispensed into a clean micro titer plate for subsequent assembly or short-term storage.

Before the 500mers are assembled into the final 10K bp gene, a small volume of the individual 500mers can be sampled and sequenced. The retention of 500mer samples can be used for quality control. For example, if it is found that the final gene has an error in the sequence, only the particular 500mer responsible for the error needs to be resynthesized rather than the entire library of 500mers. The final assembly can combine all the individual 500mers with the necessary PCR reagents and proceed in a thermal cycler. If desired, a robotic system, similar, for example, to the Beckman Coulter Biomek, can be integrated with the automated gene synthesizer.

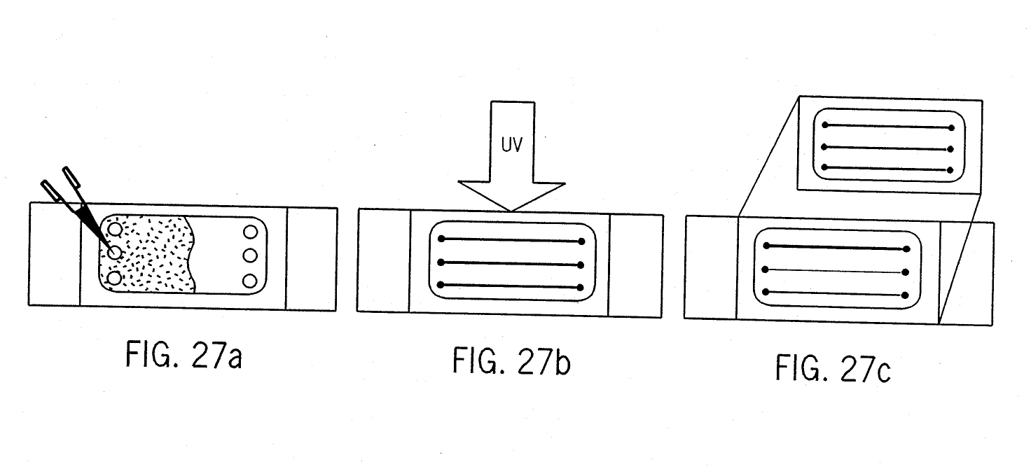

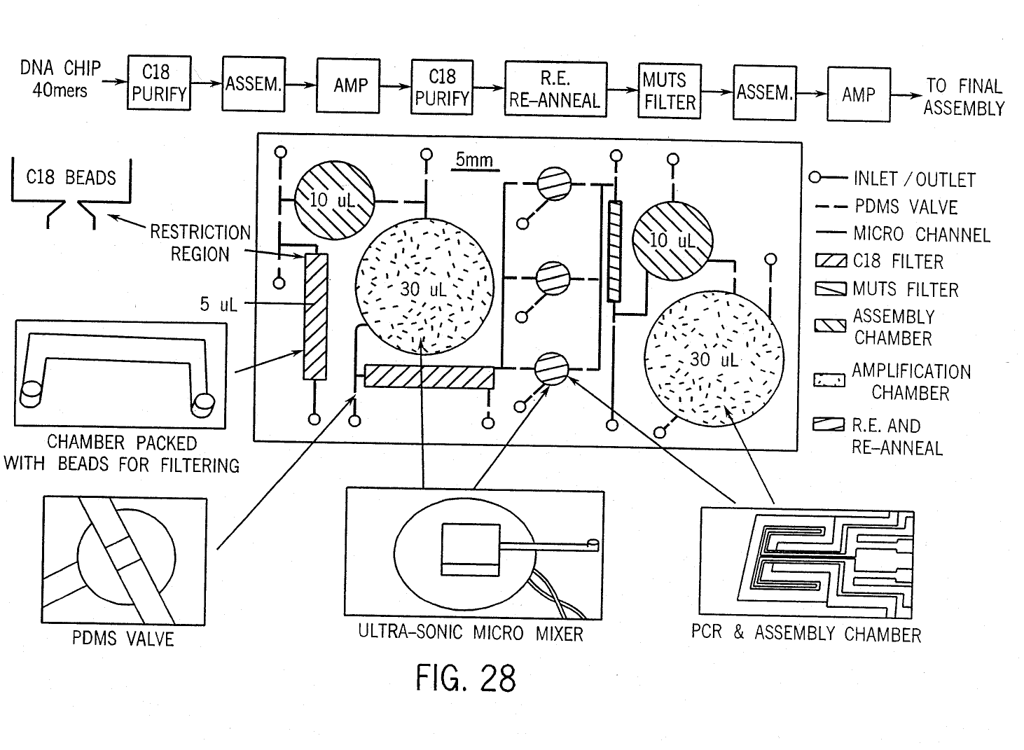

A hybrid microfluidic fabrication technology may be used to provide both flexible integration and inexpensive manufacturing, preferably using liquid phase photopolymerization methods to fabricate post-synthesis fluidics features between two glass plates, and a top PDMS (polydimethysiloxane) layer to implement fluid control valve elements. It is desirable to reduce the synthesis chamber volume to reduce reagent cost. In the synthesis chamber, the volume is preferably reduced to ˜500 nl by using capillaries as synthesis cells. However, the reduction in release volume increases the difficulty of post-synthesis fluid handling. Pipette manipulation is more difficult with smaller volumes, but microfluidics provides a more suitable approach that can be easily integrated into the post processing steps. Microfluidics can also improve the concentration of the final product by two mechanisms: the reduction of material lost due to fewer fluid transfer steps, and the reduction of final assembly reaction volume. In the robotic approach, each 500mer assembly requires up to 14 transfers (if the consensus filter is repeated 3 times) of the oligonucleotides between microtiter plates, and each of these transfers is done with pipette tips. During these handling steps, the oligonucleotides may be lost due to residual transfer volumes. The microfluidics approach greatly reduces the amount of fluid handling, and hence the reagent costs. Furthermore, the final assembly steps can be performed in smaller volumes than previously possible, resulting in higher oligonucleotide concentrations in the final product without using complicated concentration steps. Individual functional components can be implemented and integrated into a microfluidic platform. Instead of storing the eluted oligonucleotides in wells and purifying them using pipette tips (20 to 100 μL volumes), flow-through elements can be used to purify and filter the synthesis product as it is eluted from the synthesis chamber. The μFT method as illustrated in FIG. 27 starts from a universal cartridge with fluidic access ports, using simple glass chambers that have access ports on the top side. The cartridge is filled with a pre-polymer mixture (a) and a mask is placed atop for UV exposure patterning (b). The mask is removed and the unpolimerized material flushed out (c), revealing the channel network. The device is finished with a top molded PDMS layer with valve structures implemented in it. Finally, the PDMS layer is bonded to the patterned glass substrate. FIG. 28 shows a simple fluidic chip designed for the purification, assembly, and amplification of eluted oligonucleotides. This chip contains all the major components necessary for post-synthesis processing, with only one pass through the consensus filter (optimization of the consensus filter may be carried out to achieve only one pass per assembly). After the microfluidic device is fabricated, the C18 and MutS filter chambers are filled with the correct glass bead materials. The glass beads are localized in these filter elements by using a simple restriction region as shown in FIG. 28. The assembly and amplification chambers accomplish multiple tasks, including: heaters for thermal cycling, temperature sensors for thermal control, and active mixer for reagent mixing. A PDMS pinch-off valve may be incorporated with the rest of the structures for precise fluid control.

In each 10 k bp assembly, multiple microfluidic chips preferably are operated simultaneously to achieve maximum efficiency. This can be done by minimizing the chip area for each assembly process and placing multiple copies of the system on the same wafer. However, this approach is limited by the volume requirements and the useable area on a substrate. Another approach is to use a 3D stackable architecture and arrange the individual assembly chips so that they share common fluidic interconnects.

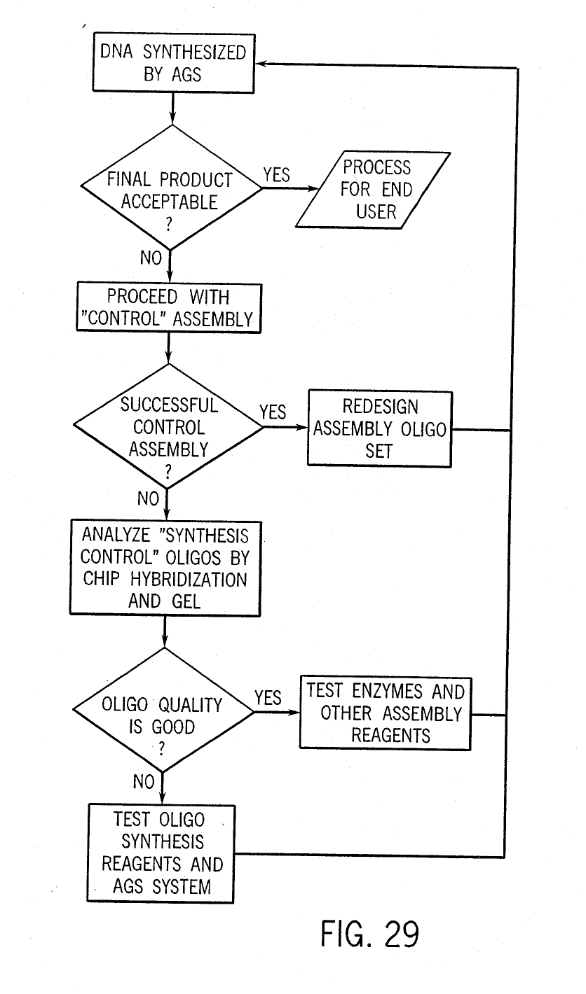

Dependent upon the chemistry utilized, many stages throughout the synthesis and assembly process can be assayed for quality control. Where photorelease chemistry is utilized, this allows for a spatial and temporal release of oligonucleotides. Therefore, it is possible to synthesize and leave a variety of “control” oligonucleotides tethered to each chip. A diagram of a control process is shown in FIG. 29. If assembly of the target gene is unsuccessful, then the “control” set can be used to determine the precise step at which failure occurred. For example, a set of “control-assembly” oligonucleotides that successfully hybridize may initially be released and can flow through the region. If no assembly of this positive control occurs, then step-wise analysis of the process can begin. However, if the control oligonucleotides are successful in assembly, this implies that the target oligonucleotides themselves may be faulty and not efficient at assembly. At this point the bioinformatics software may be utilized to produce other oligonucleotide set options to attempt a re-assembly. In addition, other “control” oligonucleotides can also be included to aid in subsequent analysis. Assuming that “control-assembly” reaction fails, then a “control-synthesis” oligonucleotide may undergo hybridization to confirm oligonucleotide identity. This experiment would thereby ensure that the instrumentation and software for DNA synthesis and placement is in proper order. However, a positive hybridization result does not conclusively indicate that the identity of an oligonucleotide population is fully correct since wild-type truncated oligonucleotides may still be successful for hybridization. For example, if the target sequence to be synthesized were a sequence of several thymine bases followed by two adenine bases (TTTTTTAA), hybridization would likely still occur with the complementary anti-sense oligonucleotide (AAAAAATT) even if the major constituent were TTTTTT (truncate). In essence, it is the forgiving nature of hybridization that causes this method not to be precise enough for the purpose of verifying the amount of full-length oligonucleotide synthesized. For that reason, the “control” hybridized chip may be stripped and the “control-synthesis” oligonucleotide eluted. This product may then be quantitated using mass spectrometry and/or gel electrophoresis to reveal the amount and quality of DNA produced.

There is currently great interest in the use of small molecule microarrays and high throughput identification of new bioactive compounds. Indeed, it is hoped that microarrays of ligands will accelerate chemical genomics in much the same way DNA microarrays have accelerated genomics. The small molecule microarrays can be formed either by physical spotting of compounds into arrays with robotics, assembly of DNA/RNA-small molecule conjugates into DNA arrays, or by in situ synthesis. A new approach to in situ synthesis is the use of photolabile protecting group chemistry for use in light directed combinatorial synthesis of small molecule arrays.

The use of light-directed combinational chemistry has thus far been limited to the synthesis of linear polymers (DNA, polypeptides, etc.) due primarily to the lack of photolabile protecting groups that allow the independent, selective deprotection of multiple protecting groups on the same molecular framework. The ability to independently cleave multiple protecting groups using light would open the door for in situ light directed combinatorial chemistry to build drug-like small molecule libraries in arrays with the MAS. Although several approaches can be envisioned to solve this problem, many suffer drawbacks that make them unattractive. One approach involves the development of protecting groups that are sensitive to different wavelengths of light, and another uses photo-generated cleavage reagents. The former approach has difficulties associated with specificity of cleavage and demands specialized light sources; the latter suffers from a loss of spatial resolution due to the generation of diffusible chemical reagents. A preferred approach is a multiple orthogonal safety-catch photolabile (SCPL) protecting group that can be independently photocleavable with a 365 nm light source through the use of a chemical pre-activation step that converts a photo-inert protecting group to a photocleavable group. These latent photocleavable protecting groups enable a large variety of small molecule combinatorial chemistry to be accomplished using a MAS modified to allow the introduction of many independent reagents during the diversity introduction steps in the synthesis. In combination with a surface sensitive method for imaging the binding of unlabelled proteins to small molecule arrays, this platform enables high throughput (up to >10000 compounds/chip) synthesis and screening of small molecule combinatorial libraries to identify library members that selectively bind to proteins.

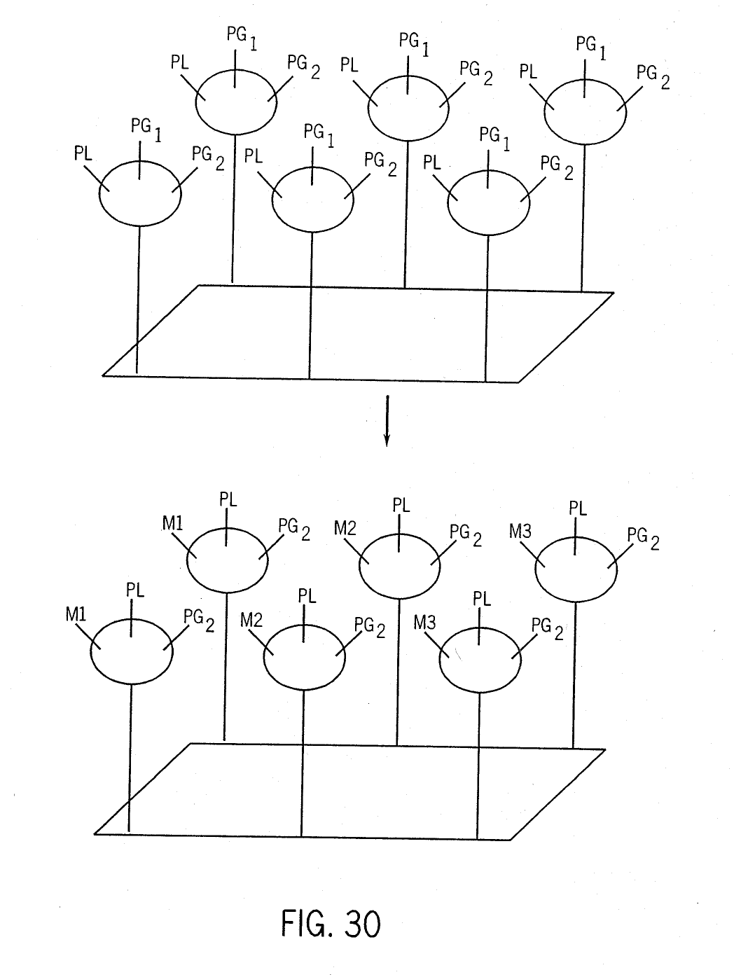

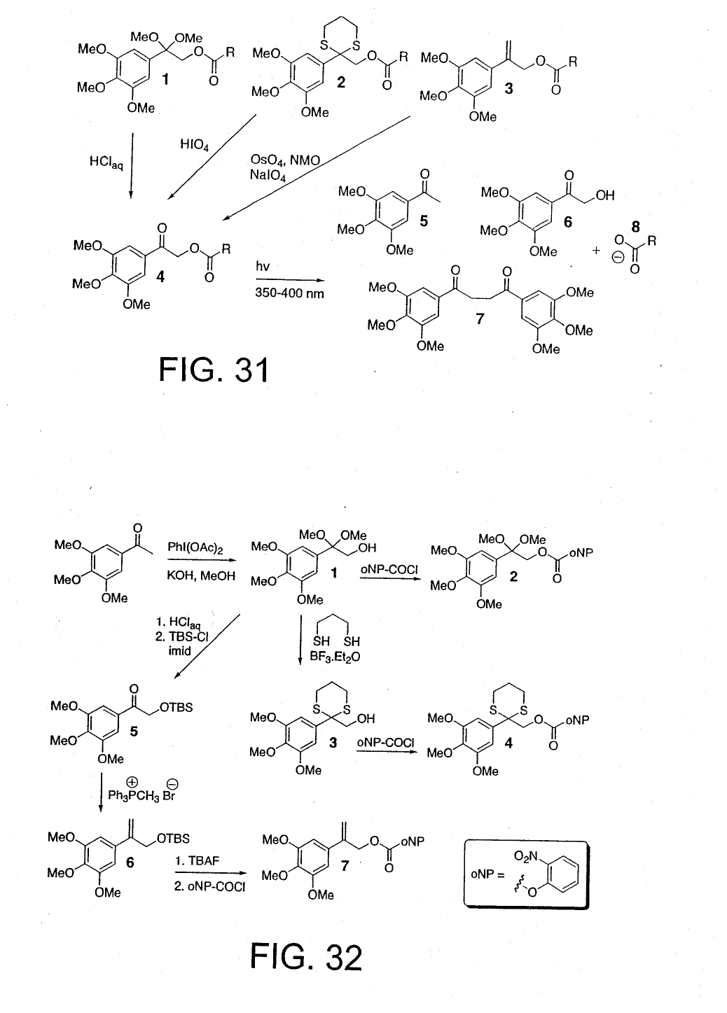

In this approach, as illustrated with reference to FIGS. 30 and 31, a suitably protected scaffold molecule is covalently tethered to a glass slide via a flexible linker. In the first cycle of combinatorial synthesis, one (of several independent) protecting groups is photochemically removed from a subset of the pixels on the slide, unveiling a reactive group on the scaffold molecule. A monomer with suitable reactivity to react with this group will be added to the surface of the array, adding diversity to a selected set of pixels, and this process is repeated with additional photodeprotection and monomer coupling cycles until all members of the array have been derivitized at the first position. A chemical activation step will then convert a second (photochemically unreactive) protecting group on the scaffold into a photocleavable group, enabling a second round of diversification. Third and fourth rounds are conducted as appropriate for the scaffold molecule. The key developments are a series of efficient, orthogonal SCPL-protecting groups for attachment to the scaffolds, and analytical methods to detect binding of biomolecules to small molecule microarrays and ultimately validation of the approach in biological screens. The phenacyl group is a preferred core structure in the SCPL-protecting groups as the mechanism of photocleavage depends upon the presence of an aryl ketone that undergoes photoexcitation to a triplet diradicaloid excited and subsequently cleaves. The ketone group is readily masked in multiple latent forms that are photoinert and can be converted to the ketone at the required time through chemical deprotection. Additionally, these groups need not contain any chiral centers, simplifying synthesis and characterization. These trimethoxyphenacyl derivatives have an absorption maximum at ˜375 nm which extends into the visible range, allowing the possibility of deprotections at both 360 nm and 400 nm, either directly or through the use of a sensitizer.

A first scheme (Scheme 1) as shown in FIG. 31 has three potential SCPL-protecting groups and conditions for orthogonal activation of each of the SCPL-protecting groups. The latent ketone in S1-1 is protected as a dimethoxy ketal that can be hydrolyzed to the ketone under mild acidic conditions. S1-2 has a dithiane masking the ketone that can be deprotected with periodate. S1-3 has the ketone masked as an alkene that can be oxidatively cleaved by treatment with OsO4, N-methylmorpholine-N-oxide and periodate. All of these SCPL-protecting may be converted to the trimethoxyphenacyl group S1-4, allowing photocleavage at long wavelengths.

At least three orthogonal SCPL-protecting groups can be synthesized. Along with the parent photolabile group, this provides four independent orthogonal photolabile protecting groups (direct photodeprotection plus three safety catch). The SCPL-protecting groups need only be orthogonal to one another within a linear sequence of activation and cleavage conditions, and thus each group need not be fully orthogonal to all others. A synthetic route is outlined in FIG. 32 and begins with commercially available trimethoxyacetophenone. Oxidation with diacetoxyiodobenzene in methanolic KOH directly provides the hydroxyl ketal S2-1. Conversion of S2-1 to the o-nitrophenyl (oNP) carbonate S2-2 provides the first reagent for introduction of a safety catch photolabile protecting group into amines and alcohols. The hydroxylketal S2-1 can be converted to the dithiane S2-3 with propanedithiol under Lewis acid catalysis. Conversion to the oNP-carbonate S2-4 provides a second reagent for introduction of a SCPL-protecting group onto amines and alcohols. Alternatively, the hydroxylated ketal can be hydrolysed to the ketone, protected with TBS-C1 and converted to the alkene S2-6 with a Wittig olefination. The alkene S2-6 can subsequently be deprotected and converted to the oNP-carbonate S2-7, providing a third reagent for the introduction of a SCPL-protecting group onto amines and alcohols.

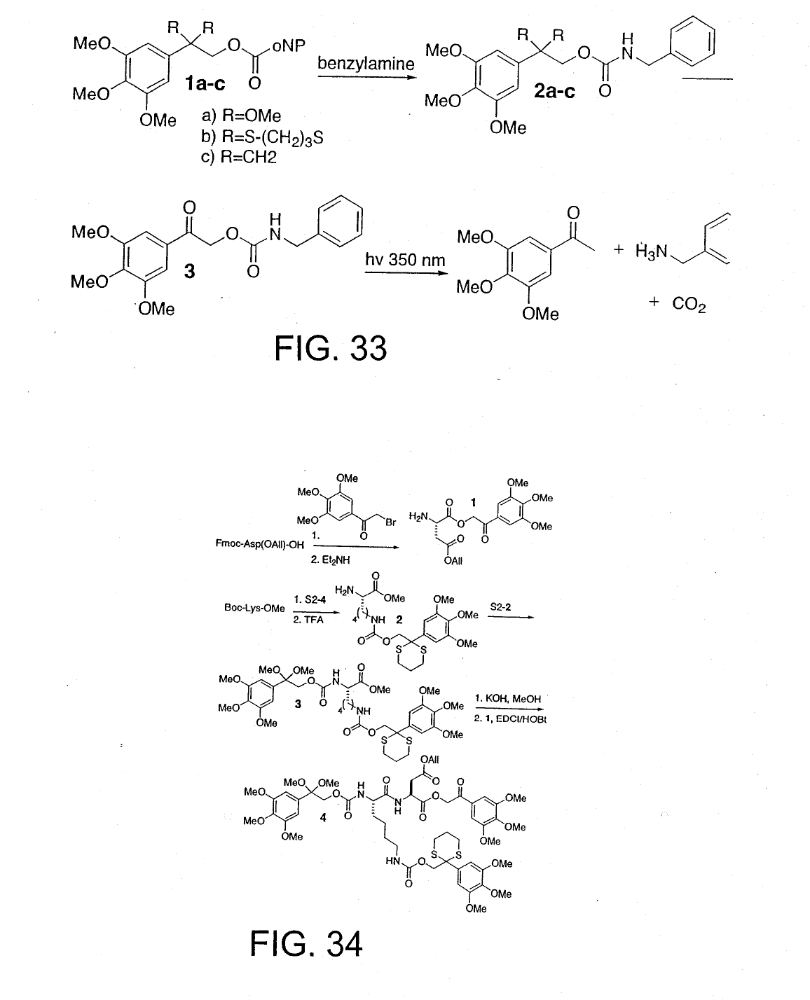

To provide a set of reagents, S2-1, S2-3 and S2-6 are converted to the active carbonates S2-2, S2-4, S2-7 for introduction into scaffold molecules. It should also be noted that S2-1, S2-3 and S2-6 can also be converted to esters for the protection of carboxylic acids. To characterize each of the SCPL-protecting groups, a series of protected benzylamines S3-1 are produced as shown in FIG. 33.

A suitably protected scaffold may be used to test up to three orthogonal SCPL-protecting groups. One scaffold may be based upon the dipeptide Lys-Glu. A synthetic route to this scaffold is shown in FIG. 34. Fmoc-Asp(OA11)-OH is protected as the trimethoxy phenacyl ester with triethoxyphenacyl bromide and deprotected with diethylamine to give the amine S4-1. Boc-Lys-OMe is acylated with the dithiane carbonate S2-4 and deprotected with trifluoroacetic acid to give amine S4-2 which is subsequently acylated with S2-2 to give urethane S4-3. Hydrolysis of the methyl ester and coupling to amine 1 with EDCl/HOBt provides amine 4 for testing the orthogonality of the SCPL-protecting groups.

Compound 4 is subjected to UV photolysis to deprotect the a-carboxyl of aspartic acid and coupled to benzylamine with PyAOP. Treatment with 5% trifluoroacetic acid can unveil the photolabile group protecting the α-amine of lysine. Photodeprotection and coupling with benzoyl chloride will cap the amine. Deprotection of the dithiane with periodate will activate the final safety-catch for photolysis and coupling to benzoyl chloride. The allyl ester of S4-4 can be deprotected with Pd to allow covalent attachment to amine terminated glass slides. Various fluorescent dyes may be used on the three sites on the Lys-Asp dipeptide for independent, orthogonal deprotection of the SCPL-protecting groups. Using a set of orthogonal SCPL-protecting groups, biologically interesting scaffolds can be chosen for the creation and screening of microarrayed combinatorial libraries through in situ synthesis.

It is understood that the invention is not limited to the embodiments set forth herein as illustrative, but embraces all such forms thereof as come within the scope of the following claims.

What is claimed is:

1. A method for the generation of a long double stranded DNA target sequence comprising:
(a) synthesizing a set of oligonucleotides that contain sections of the target sequence, each oligonucleotide attached to a support by a cleavable linker;
(b) cleaving the linker to release selected oligonucleotides in a desired sequence, bringing the released oligonucleotides together, and joining selected oligonucleotides to form a set of subsequences which are parts of the desired target sequence; and
(c) assembling the subsequences to form the desired target sequence.

2. The method of claim 1 further including, before the step of synthesizing, identifying the set of subsequences that can be assembled to form the target sequence, and further identifying the set of oligonucleotides that can be assembled together to form each subsequence.

3. The method of claim 1 further including carrying out error correction on the oligonucleotides and on the subsequences.

4. The method of claim 3 wherein the error correction is carried out by DNA coincidence filtering.

5. The method of claim 4 wherein the DNA coincidence filtering is carried out by passing double stranded oligonucleotides and subsequences through a filter containing MutS protein to bind DNA duplexes containing mismatched bases while allowing error free duplexes to pass through.

6. The method of claim 1 wherein the synthesized oligonucleotides are held to the support by photocleavable linkers and wherein releasing selected oligonucleotides comprises illuminating one or more areas of the support containing the selected oligonucleotides to photocleave the linkers holding the oligonucleotides to the support.

7. The method of claim 1 wherein the synthesized oligonucleotides are held to the support by chemically labile linkers and wherein releasing selected oligonucleotides comprises applying a reagent that cleaves the linker to one or more areas of the support containing the selected oligonucleotides to cleave the linkers holding the oligonucleotides to the support.

8. The method of claim 1 wherein the oligonucleotides are synthesized with primer sequences at their ends, the method further including the step of conducting polymerase chain reaction amplification of the oligonucleotides after release of the oligonucleotides from the support and before assembling the oligonucleotides to form the subsequences.

9. The method of claim 1 further including carrying out polymerase chain reaction amplification of the subsequences before assembly of the subsequences into the target sequence.

10. The method of claim 1 wherein synthesizing the set of oligonucleotides is carried out in a maskless array synthesizer having a reaction chamber in which DNA synthesis reactions are performed on the support with an active surface in which arrays of different oligonucleotides are formed, a flow cell enclosing the active surface of the support and having ports for supplying reagents into the flow cell which can be flowed over the active surface of the support, a DNA synthesizer reagent supply connected to supply reagents to the flow cell, and an image former for providing a high precision, array light image projected onto the substrate active support.

11. A method for the generation of nucleotides having a desired sequence comprising:
(a) synthesizing a set of double stranded nucleotides that are intended to contain the desired sequence;
(b) carrying out coincidence filtering error correction on the nucleotides by passing double stranded nucleotides through a filter that binds DNA duplexes containing mismatched bases while allowing error free duplexes to pass through.

12. The method of claim 11 wherein the filter contains MutS protein to bind DNA duplexes containing mismatched bases.

13. The method of claim 11 wherein the synthesized nucleotides are held to a support by photocleavable linkers and wherein releasing selected nucleotides comprises illuminating one or more areas of the support containing the selected nucleotides to photocleave the linkers holding the nucleotides to the support.

14. The method of claim 11 wherein the synthesized nucleotides are held to the support by chemically labile linkers and wherein releasing selected nucleotides comprises applying a reagent that cleaves the linker to one or more areas of the support containing the selected nucleotides to cleave the linkers holding the nucleotides to the support.

15. The method of claim 11 wherein the nucleotides are synthesized with primer sequences at their ends, the method further including the step of conducting polymerase chain reaction amplification of the nucleotides.

16. The method of claim 11 wherein synthesizing a set of nucleotides is carried out in a maskless array synthesizer having a reaction chamber in which DNA synthesis reactions are performed on a support with an active surface in which arrays of different nucleotides are formed, a flow cell enclosing the active surface of the support and having ports for supplying reagents into the flow cell which can be flowed over the active surface of the support, a DNA synthesizer reagent supply connected to supply reagents to the flow cell, and an image former for providing a high precision, array light image projected onto the support active surface.

17. A method for the generation of a long double stranded DNA target sequence comprising:
(a) synthesizing a set of oligonucleotides that contain sections of the target sequence, each oligonucleotide attached to a support by a cleavable linker, wherein synthesizing is carried out in a maskless array synthesizer having a reaction chamber in which DNA synthesis reactions are performed on the support with an active surface in which arrays of different oligonucleotides are formed, a flow cell enclosing the active surface of the support and having ports for supplying reagents into the flow cell which can be flowed over the active surface of the support, a DNA synthesizer reagent supply connected to supply reagents to the flow cell, and an image former for providing a high precision, array light image projected onto the support active surface;
(b) cleaving the linker to release selected oligonucleotides in a desired sequence, bringing the released oligonucleotides together, and joining selected oligonucleotides to form a set of subsequences which are parts of the desired target sequence; and
(c) assembling the subsequences to form the desired target sequence.

18. The method of claim 17 further including, before the step of synthesizing, identifying the set of subsequences that can be assembled to form the target sequence and identifying the set of oligonucleotides that can be assembled to form each subsequence.

19. The method of claim 17 further including carrying out error correction on the oligonucleotides and on the subsequences.

20. The method of claim 19 wherein the error correction is carried out by DNA coincidence filtering.

21. The method of claim 20 wherein the DNA coincidence filtering is carried out by passing double stranded oligonucleotides and subsequences through a filter containing MutS protein to bind DNA duplexes containing mismatched bases while allowing error free duplexes to pass through.

22. The method of claim 17 wherein the synthesized oligonucleotides are held to the support by photocleavable linkers and wherein releasing selected oligonucleotides comprises illuminating one or more areas of the support containing the selected nucleotides to photocleave the linkers holding the oligonucleotides to the support.

23. The method of claim 17 wherein the synthesized oligonucleotides are held to the support by chemically labile linkers and wherein releasing selected oligonuceotides comprises applying a reagent that cleaves the linker to one or more areas of the support containing the selected oligonucleotides to cleave the linkers holding the oligonucleotides to the support.

24. The method of claim 17 wherein the oligonucleotides are synthesized with a primer sequences at their ends, the method further including the step of conducting polymerase chain reaction amplification of the oligonucleotides after release of the oligonucleotides from the support and before assembling the oligonucleotides to form the subsequences.

25. The method of claim 17 further including carrying out polymerase chain reaction amplification of the subsequences before assembly of the subsequences into the target sequence.
